(19) United States
(12) Patent Application Publication
(10) Pub. No.: US 2024/0100088 A1
(43) Pub. Date: Mar. 28, 2024

(54) NOVEL FORMULATION FOR TREATING COPPER METABOLISM-ASSOCIATED DISEASES OR DISORDERS

(71) Applicant: Alexion Pharmaceuticals, Inc., Boston, MA (US)

(72) Inventors: Justin Lockheart Burt, Shelton, CT (US); David Jenson Chen, Boston, MA (US); Todd Anthony Stutzman, Overland Park, KS (US); Allissa Robin Kerner, Kansas City, MO (US); Joseph Michael Schnitz, Kansas City, MO (US); Raj Ramnik Jain, Overland Park, KS (US)

(73) Assignee: Alexion Pharmaceuticals, Inc., Boston, MA (US)

(21) Appl. No.: 18/272,308

(22) PCT Filed: Jan. 31, 2022
(86) PCT No.: PCT/US2022/014571

§ 371 Date: Jul. 13, 2023

Related U.S. Application Data
(60) Provisional application No. 63/143,897, filed on Jan. 31, 2021.
(60) Provisional application No. 63/229,358, filed on Aug. 4, 2021.

(51) Int. Cl. A61K 33/24; A61K 9/20; A61K 9/28

(52) U.S. Cl. CPC A61K 33/24; A61K 9/2054; A61K 9/2095; A61K 9/2893

(57) ABSTRACT

This disclosure relates to novel formulations of bis-choline tetrathiomolybdate useful for treating a copper metabolism-associated disease or disorder, such as Wilson disease (WD). For example, this disclosure relates to low dose formulations of bis-choline tetrathiomolybdate.

### BACKGROUND OF THE DISCLOSURE

### Field of the Disclosure

This disclosure relates to novel formulations of bis-choline tetrathiomolybdate useful for treating a copper metabolism-associated disease or disorder, such as Wilson disease (WD). For example, this disclosure relates to low dose formulations, such as mini-tablets, of bis-choline tetrathiomolybdate, and capsules, sachets, stick packs, and kits comprising these formulations.

### Description of Related Art

Wilson disease (WD) is a rare, autosomal recessive disorder of impaired copper (Cu) transport that results in pathological Cu accumulation. In WD, mutations in the ATP7B gene result in deficient production of adenosine triphosphatase 2 (ATPase2), which in turn leads to impaired biliary excretion of Cu and impaired incorporation of Cu into ceruloplasmin (CP), a serum ferroxidase, which, in healthy humans, contains greater than 95% of the Cu found in plasma. Consequently, there is an increase of Cu in liver, brain, and other tissues with resultant organ damage and dysfunction. Initial signs and symptoms of WD are predominantly hepatic, neurologic, or psychiatric, but patients often develop combined hepatic and neuropsychiatric disease. Untreated or inadequately treated patients have progressive morbidity, and mortality is usually secondary to hepatic cirrhosis. Other causes of death associated with WD include hepatic malignancy and neurologic deterioration with severe inanition.

The current treatments for WD are the general chelator therapies D-penicillamine and trientine, which chelate Cu and promote urinary Cu excretion, and zinc (Zn), which blocks dietary uptake of Cu through upregulation of intestinal metallothionein. The currently available drugs have high rates of treatment discontinuation due to tolerability and efficacy issues as well as non-adherence to the treatment regimen. For example, the currently available drugs require frequent dosing (e.g., 2 to 4 times per day) and must be taken in a fasted state for each dose. Their adverse event (AE) profiles and complicated dosing regimens lead to poor treatment compliance and high rates of treatment failure, a major concern in WD, which requires life-long treatment.

Bis-choline tetrathiomolybdate (also known as BC-TTM, tiomolibdate choline, tiomolibdic acid, and WTX101) is an investigational, oral, first-in-class copper-protein-binding molecule being developed for the treatment of WD and has been described in detail in International Publication No. WO 2019/110619 (incorporated by reference herein in its entirety). BC-TTM has the following structure:

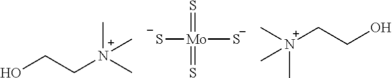

There exists a need in the art for improved drug delivery systems for delivery of BC-TTM for use in patient populations having variable dosing needs.

### SUMMARY OF THE DISCLOSURE

One aspect of the disclosure provides a mini-tablet formulation comprising bis-choline tetrathiomolybdate in an amount in the range of about 1.00 mg to about 1.50 mg.

Another aspect of the disclosure provides a mini-tablet formulation comprising:
bis-choline tetrathiomolybdate in an amount of about 1.25 mg;
about 25% (by weight based on the weight of mini-tablet core) of a buffer;
about 66% (by weight based on the weight of mini-tablet core) of a filler component;
about 0.75% (by weight based on the weight of mini-tablet core) of the lubricant component.

Another aspect of the disclosure provides a unit dose comprising one or more of the mini-tablets of the disclosure. In certain embodiments, the unit dose of the disclosure comprises two or more of the mini-tablets of the disclosure.

Another aspect of the disclosure provides a capsule, a sachet, or a stick pack comprising the unit dose of the disclosure as described herein. Another aspect of the disclosure provides a unit dose dispenser configured to dispense a unit dose of the disclosure as described herein.

Another aspect of the disclosure provides methods for treating a copper metabolism-associated disease or disorder in a subject. Such methods include administering to the subject one or more mini-tablets of the disclosure as described herein or a unit dose of the disclosure as described herein. In certain embodiments, the unit dose of the disclosure can be provided in a unit dose container, such as a capsule, a sachet, a stick pack, or dispensed from the unit dose dispenser as described herein.

Another aspect of the disclosure provides use of one or more of mini-tablet of the disclosure as described herein or a unit dose of the disclosure as described herein for the manufacture of a medicament. In certain embodiments, the unit dose can be provided in a unit dose container, such as a capsule, a sachet, a stick pack, or dispensed from the unit dose dispenser as described herein. In certain embodiments, the use is for a manufacture of a medicament for treating a copper metabolism-associated disease or disorder in a subject.

These and other features and advantages of the claimed invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the formulations and methods of the disclosure, and are incorporated in and constitute a part of this specification. The drawings illustrate one or more embodiment(s) of the disclosure and, together with the description, serve to explain the principles and operation of the disclosure.

FIG. 1 illustrates the stability of the mini-tablet formulation of the disclosure (F2G2; circles, solid line) and a comparative formulation (5 mg; triangles, dashed line) after 4 weeks of storage. Top chart shows the concentration of total impurities (%) in the formulation over time; bottom chart shows the concentration of BC-TTM (%) in the formulation over time.

FIG. 2 illustrates the stability of the mini-tablet formulations of the disclosure, F2G2 (circles, solid line) and F1G2 (squares ; dotted line), after 4 weeks of storage. Top chart shows the concentration of total impurities (%) in the formulation over time; bottom chart shows the concentration of BC-TTM (%) in the formulation over time.

### DETAILED DESCRIPTION OF THE DISCLOSURE

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and formulations described herein can be configured by the person of ordinary skill in the art to meet the desired need. The present disclosure provides improvements in treating copper metabolism-associated diseases or disorders.

Wilson disease (also called hepatolenticular insufficiency) is an inherited disease of copper transport. Wilson disease is caused by a variety of genetic mutations in the Cu-loading enzyme ATP7B (in humans). ATP7B facilitates the transfer of Cu to CP and Cu-excretion via biliary canaliculi. The resulting defect in the hepatic excretory pathway leads to accumulation of copper in tissues such as the liver, kidneys, the central nervous system/brain, and the cornea, and copper levels remain elevated without treatment. Specifically, copper accumulation exceeds the capacity of CP, giving rise to free, non-ceruloplasmin bound copper (“NCC”) circulating in the blood and accumulating in tissues and organs. This NCC may loosely bind with plasma proteins (such as, for example, albumin, transcuprein, and low molecular weight peptides or amino acids) to form complexes (“labile-bound copper” or “LBC”).

In certain embodiments of the methods and uses of the disclosure as described herein, the copper metabolism associated disease or disorder is Wilson disease.

In certain embodiments, the copper metabolism associated disease or disorder is copper toxicity (e.g., from high exposure to copper sulfate fungicides, ingesting drinking water high in copper, overuse of copper supplements, etc.). In certain embodiments, the copper metabolism associated disease or disorder is copper deficiency, Menkes disease, or aceruloplasminemia. In certain embodiments, the copper metabolism associated disease or disorder is at least one selected from academic underachievement, acne, attention-deficitihyperactivity disorder, amyotrophic lateral sclerosis (ALS), atherosclerosis, autism, Alzheimer's disease, Candida overgrowth, chronic fatigue, cirrhosis, depression, elevated adrenaline activity, elevated cuproproteins, elevated norepinephrine activity, emotional meltdowns, fibromyalgia, frequent anger, geriatric-related impaired copper excretion, high anxiety, hair loss, hepatic disease, hyperactivity, hypothyroidism, intolerance to estrogen, intolerance to birth control pills, Kayser-Fleischer rings, learning disabilities, low dopamine activity, multiple sclerosis, neurological problems, oxidative stress, Parkinson's disease, poor concentration, poor focus, poor immune function, ringing in ears, allergies, sensitivity to food dyes, sensitivity to shellfish, skin metal intolerance, skin sensitivity, sleep problems, and white spots on fingernails.

The present disclosure advantageously provides low dose formulations, such as mini-tablets, comprising bis-choline tetrathiomolybdate (BC-TTM) that can be administered in varying doses to a patient population where there is an ongoing need for monitoring and dose adjustment throughout a patient's life. In particular, a patient's dose can remain constant or can be adjusted to maintain a therapeutic level of BC-TTM and satisfactory copper levels. In some embodiments, the disclosure further provides a capsule, a sachet, or a stick pack comprising one or more of the mini-tablets that allows for administration of a specific dose of BC-TTM based on a patient's need. In some other embodiments, the disclosure further provides a unit dose dispenser configured to dispense a unit dose of mini-tablets.

In some embodiments, the mini-tablet formulation disclosed herein comprises BC-TTM in an amount of about 1.00 mg to about 1,50 mg. For example, BC-TTM may be present in an amount in the range of about 1.10 mg to about 1,40 mg, or about 1.15 mg to about 1,35 mg, or about 1.20 mg to about 1,30 mg, or about 1.22 mg to about 1.28 mg, or about 1.23 mg to about 1.27 mg, or about 1.24 mg to about 1.26 mg. In some embodiments, the amount is in the range of about 1.00 mg to about 1.25 mg. In some embodiments, the mini-tablet formulation disclosed herein comprises BC-TTM in an amount of about 1.25 mg.

In some embodiments, the mini-tablet formulation disclosed herein comprises about 5% to about 10% (by weight based on the weight of mini-tablet core, i.e., the weight of the tablet excluding the coating) of BC-TTM. In some embodiments, the mini-tablet formulation comprises about 5%, about 5,5%, about 6.0%, about 6.5%, about 7.0%, about 7.5%, about 8.0%, about 8.5%, about 9.0%, about 9.5%, or about 10% (by weight based on the weight of mini-tablet core) of BC-TTM. In particular embodiments, the mini-tablet formulation comprises about 8.33% (by weight based on the weight of mini-tablet core) of BC-TTM.

In some embodiments, the mini-tablet formulation disclosed herein comprises one or more buffers. As used herein, “buffer” refers to an excipient for maintaining the pH of a formulation. In particular embodiments, the buffer is sodium bicarbonate (NaHCO₃). Sodium bicarbonate provides superior stabilization of BC-TMM and advantageously allows a formulation of BC-TMM that does not require a disintegrant for stabilization.

In some embodiments, the mini-tablet formulation comprises about 20% to about 30% (by weight based on the weight of mini-tablet core) of the buffer. For example, buffer may be present in the range of about 22 wt % to about 28 wt %, or about 23 wt % to about 27 wt %, or about 24 wt % to about 26 wt %, or about 20 wt % to about 25 wt %, or about 25 wt % to about 30 wt %, based on the weight of mini-tablet core. In some embodiments, the mini-tablet formulation comprises about 20 wt %, about 21 wt %, about 22 wt %, about 23 wt %, about 24 wt %, about 25 wt %, about 26 wt %, about 27 wt %, about 28 wt %, about 29 wt %, or about 30 wt %, based on the weight of mini-tablet core, of the buffer. In particular embodiments, the mini-tablet formulation comprises about 25 wt %, based on the weight of mini-tablet core, of the buffer.

In some embodiments, the mini-tablet formulation comprises BC-TTM and sodium bicarbonate present in a weight ratio in a range of about 10:90 to 40:60 (for example in a range of about 20:80 to 30:70). In some embodiments, the mini-tablet formulation comprises BC-TTM and sodium bicarbonate in about a 10:90 ratio, about a 20:80 ratio, about a 25:75 ratio, about a 30:70 or about a 40:60 ratio. In some embodiments, the mini-tablet formulation comprises BC-TTM and sodium bicarbonate in a weight ratio of about 25:75.

In some embodiments, the mini-tablet formulation disclosed herein comprises a filler component. In particular embodiments, the filler component is tribasic calcium phosphate, dibasic calcium phosphate, lactose monohydrate, lactose anhydrous, spray-dried lactose, microcrystalline cellulose, powdered cellulose, silicified microcrystalline cellulose, starch, pregelatinized starch or combinations thereof. In particular embodiments, the filler component is microcrystalline cellulose. In some embodiments, the mini-tablet formulation comprises about 60% to about 70% (by weight based on the weight of mini-tablet core) of the filler component. For example, the filler component may be present in the range of about 62 wt % to about 70 wt %, or about 63 wt % to about 69 wt %, or about 64 wt % to about 68 wt %, or about 65 wt % to about 67 wt %, based on the weight of mini-tablet core. In some embodiments, the mini-tablet formulation comprises about 60 wt %, about 61 wt %, about 62 wt %, about 63 wt %, about 64 wt %, about 65 wt %, about 66 wt %, about 67 wt %, about 68 wt %, about 69 wt %, or about 70 wt %, based on the weight of mini-tablet core, of the filler component. In particular embodiments, the mini-tablet formulation comprises about 65 wt %, based on the weight of mini-tablet core, of the filler component. In particular embodiments, the mini-tablet formulation comprises about 66 wt %, based on the weight of mini-tablet core, of the filler component.

In some embodiments, the mini-tablet formulation disclosed herein comprises a lubricant component. In particular embodiments, the lubricant component is sodium stearyl fumarate, glyceryl behenate (i.e., Compritol 888 ATO), glyceryl monostearate, stearic acid, magnesium stearate, calcium stearate, hydrogenated vegetable oil, polyethylene glycol (PEG) 4000-6000, sodium lauryl sulfate (SLS), or combinations thereof. In particular embodiments, the lubricant component is sodium stearyl fumarate (sodium (E)-4-octadecoxy-4-oxobut-2-enoate). In particular embodiments, the lubricant component is a hydrophilic lubricant. In some embodiments, the mini-tablet formulation comprises about 0.5% to about 1% (by weight based on the weight of mini-tablet core) of the lubricant component. For example, the lubricant component may be present in the range of about 0.6 wt % to about 0.9 wt %, or about 0.65 wt % to about 0.85 wt %, or about 0.7 wt % to about 0.8 wt %, or about 0.72 wt % to about 0.78 wt %, or about 0.73 wt % to about 0.77 wt %, based on the weight of mini-tablet core. In some embodiments, the mini-tablet formulation comprises about 0.5 wt %, about 0.6 wt %, about 0.7 wt %, about 0.8 wt %, about 0.9 wt % or about 1.0 wt %, based on the weight of mini-tablet core, of the lubricant component. In particular embodiments, the mini-tablet formulation comprises about 0.75 wt %, based on the weight of mini-tablet core, of the lubricant component.

In some embodiments, the mini-tablet further comprises a coating on the outer surface of the formulation. For example, the coating may be an outer surface of the mini-tablet's core that comprises bis-choline tetrathiomolybdate and, if present, the buffer, the filler component, and/or the lubricant component. In some embodiments, the coating may comprise a seal coating, a sub-coating, an enteric coating, or a combination thereof. In some embodiments, the seal coating comprises a hydrophobic material, such as for example carnauba wax. In some embodiments, the sub-coating comprises a hydrophilic material. In some embodiments, the enteric coating comprises a methacrylic acid copolymer. In some embodiments, the coating may comprise at least two layers (e.g., three layers). In some embodiments, the coating comprises Carnauba Wax Powdered as a seal coating, Opadry 200 Clear 203A190001 as a sub-coating, or Acryl-EZE White as an enteric coating, or a combination thereof.

Surprisingly, the mini-tablet formulations of the disclosure as described herein maintain high level of purity after a prolonged storage. For example, in certain embodiments, the mini-tablet formulation of the disclosure as described herein comprises no more than about 0.7%, or no more than about 0.6%, or no more than about 3.5%, or no more than about 3.25%, or no more than about 3%, or no more than about 2.75%, or no more than about 2.5%, or in the range of about 2% to about 3% of total impurities at 4 weeks of storage at about 25° C. at about 60% relative humidity as determined by HPLC.

Common impurities observed in BC-TTM formulations are molybdenum impurities, including, for example, TM0, TM1, TM2, and TM3.

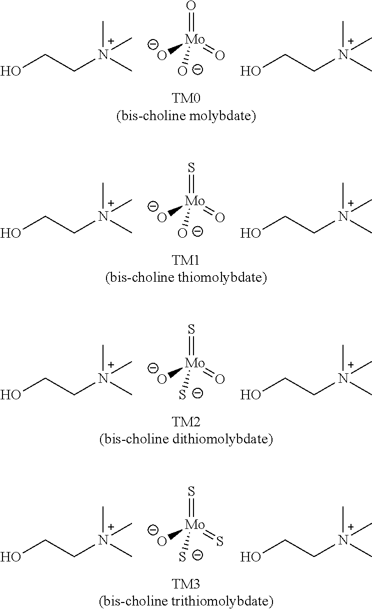

Other common impurities include polymeric molybdenum impurities, such as Dimer S6 and Dimer S7 shown below.

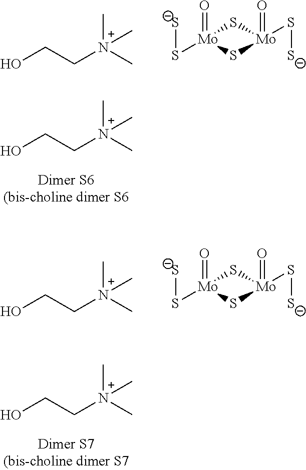

In certain embodiments, the mini-tablet formulation of the disclosure as described herein comprises less than about 2%, or less than about 1,8%, or less than about 1.7%, or less than about 1.6%, or in the range of about 1% to about 2% of total molybdenum impurities, wherein the molybdenum impurities are selected from one or more of TM0, TM1, TM2, and TM3, at 4 weeks of storage at about 25° C. at about 60% relative humidity as determined by HPLC.

In certain embodiments, the mini-tablet formulation of the disclosure as described herein comprises no more than about 0.7%, or no more than about 0.6%, or no more than about 0.5%, or no more than about 0.4%, or no more than about 0.3%, or in the range of about 0.1% to about 0.5% of polymeric molybdenum impurities at 4 weeks of storage at about 25° C. at about 60% relative humidity as determined by HPLC.

In certain embodiments, the mini-tablet formulation of the disclosure as described herein has low levels of TM3 impurity after a prolonged storage. In certain embodiments, the mini-tablet formulation of the disclosure comprises less than about 1.3%, or less than about 1,2%, or less than about 1.1%, or less than about 1%, or in the range of about 0.8 to about 1% of TM3 impurity at 4 weeks of storage at about 25° C. at about 60% relative humidity as determined by HPLC.

In certain embodiments, the mini-tablet formulation of the disclosure as described herein has low levels of Dimer S6 impurity after a prolonged storage. In certain embodiments, the mini-tablet formulation of the disclosure comprises less than about 0.3%, or less than about 0.2%, or less than about 0,1%, or in the range of about 0.08 to about 0.12% of Dimer S6 impurity at 4 weeks of storage at about 25° C. at about 60% relative humidity as determined by HPLC.

In some embodiments, the disclosure further provides a unit dose comprising one or more of the mini-tablets of the disclosure. In certain embodiments, the unit dose of the disclosure comprises two or more of the mini-tablets of the disclosure.

In certain embodiments, one or more of the unit doses of the disclosure can be provided in a unit dose container. Examples of suitable unit dose containers include, but are not limited to, a capsule, a sachet, a stick pack, or a unit dose dispenser. Thus, the unit dose container of the disclosure may comprise one unit dose of the disclosure. Such containers would include a capsule, a sachet, or a stick pack. The unit dose container of the disclosure may also comprise two or more of the unit doses of the disclosure. Examples of such containers include a dispenser.

In some embodiments, the unit dose container of the disclosure is configured to dispense a unit dose of mini-tablets (such as one unit dose). Such unit dose container enables patient populations having an inability to swallow tablets and capsules, such as pediatric and geriatric populations, to access and administer a dose of the mini-tablets without having to swallow a whole tablet or capsule. In some embodiments, the unit dose container is a capsule that can be opened by the patient (such as a sprinkle capsule), a sachet, or a stick pack. In some embodiments, the unit dose container is a mini-tablet dispenser, such as those commercialized by Phillips Medisize.

In some embodiments, the unit dose comprises about 2.5 mg, about 3.75 mg, about 5 mg, about 6.25 mg, about 7.5 mg, about 8.75 mg, about 10 mg, about 11.25 mg, about 12.5 mg, about 13.75 mg, about 15 mg, about 20 mg, or about 30 ma of BC-TTM. In some embodiments, the unit dose comprises about 2.5 mg, about 3,75 mg, about 4 mg, about 5 mg, about 6 mg, about 7 mg, about 8 mg, about 9 mg, about 10 mg, about 11 mg, or about 12 mg of BC-TTM. In some embodiments, the unit dose comprises about 15 mg, about 20 mg, about 25 mg, or about 30 mg, of BC-TTM. In some embodiments, the unit dose comprises about 15 mg of BC-TTM.

In some embodiments, a unit dose container, such as in an openable capsule, sachet, stick pack, provides a dose of about 5 mg to about 30 mg of BC-TTM. In some embodiments, a unit dose container provides a dose of about 2.5 mg to about 12.5 mg, e.g., about 2.5 mg, or about 5 mg, or about 10 mg, of BC-TTM. In some embodiments, a unit dose container provides a dose of about 15 mg to about 30 mg, e.g., about 15 mg, or about 20 mg, or about 30 mg, of BC-TTM.

In some embodiments, the unit dose container comprises at least about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24 of the 1.25 mg mini-tablets. In some embodiments, the unit dose container comprises 6 of the 1,25 mg mini-tablets. In some embodiments, the unit dose container comprises more than 24 of the 1.25 mg mini-tablets.

In some embodiments, the unit dose container is a mini-tablet dispenser configured to dispense a unit dose of mini-tablets comprising about 2.5 mg, about 3.75 mg, about 5 mg, about 6,25 mg, about 7.5 mg, about 8.75 mg, about 10 mg, about 11.25 mg, about 12.5 mg, about 13.75 mg, about 15 mg, about 20 mg, or about 30 mg of BC-TTM, In some embodiments, the dispenser is configured to dispense a unit dose of mini-tablets comprising about 2.5 ma, about 3.75 mg, about 4 mg, about 5 mg, about 6 mg, about 7 mg, about 8 mg, about 9 mg, about 10 mg, about 11 mg, or about 12 mg of BC-TTM. In some embodiments, the dispenser is configured to dispense a unit dose of mini-tablets comprising about 15 mg, about 20 mg, about 25 mg, or about 30 mg, of BC-TTM. In some embodiments, the dispenser is configured to dispense a unit dose of mini-tablets of about 15 mg of BC-TTM.

In some embodiments, a dispenser dispenses mini-tablets providing a unit dose of about 5 mg to about 30 mg of BC-TTM. In some embodiments, a dispenser dispenses mini-tablets providing a unit dose of about 2.5 mg to about 12.5 mg, e.g., about 2.5 mg, or about 5 mg, or about 10 mg, of BC-TTM. In some embodiments, a dispenser dispenses mini-tablets providing a unit dose of about 15 mg to about 30 mg, e.g., about 15 mg, or about 20 mg, or about 30 mg, of BC-TTM.

In some embodiments, the dispenser dispenses a unit dose of mini-tablets comprising at least about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24 of the 1.25 mg mini-tablets. In some embodiments, the dispenser dispenses a unit dose of 6 of the 1.25 ma mini-tablets. In some embodiments, the dispenser dispenses a unit dose of mini-tablets comprising more than 24 of the 1.25 mg mini-tablets.

The dispenser, in certain embodiments, is configured to dispense one unit dose of the disclosure and conveniently include more than one unit dose, such as 15, or 30, or 60 unit doses. Thus, in certain embodiments, the dispenser includes at least about 30 to about 720 of the 1.25 mg mini-tablets (e.g., a 30-day supply). In certain embodiments, the dispenser includes at least about 90 to about 360 of the 1,25 mg mini-tablets. In certain embodiments, the dispenser includes about 90, or about 18, or about 360 of the 1.25 mg mini-tablets.

As noted above, the unit dose container of the disclosure provides a convenient means for providing a dose of the mini-tablets. For example, the capsule, sachet, or stick pack is configured to be opened by the patient (e.g., such as a sprinkle capsule). Thus, in some embodiments of the methods of the disclosure, the administration comprises opening of the capsule, sachet, or stick pack or dispensing a unit dose of mini-tablets from the mini-tablet dispenser, and providing the mini-tablet contents to food (such as soft acidic food). Without being bound by a theory, it is believed that the mini-tablets could be administered together with acidic soft foods to protect the enteric coating through to the site of absorption in the gastrointestinal tract. In some embodiments, the one or more mini-tablets are administered by sprinkling the one or more mini-tablets on soft acidic foods such as applesauce or yogurt. In some embodiments, administration of one or more of the mini-tablets with food results in a statistically equivalent mean bioavailability to the one or more of the mini-tablets administered without food.

The one or more mini-tablets or the unit dose of the disclosure as described herein may be administered daily in the methods and uses of the disclosure as described herein. For example, in certain embodiments, the one or more mini-tablets or the unit dose is administered once daily. In certain embodiments of the methods and uses of the disclosure as described herein, the one or more mini-tablets or the unit dose may be administered every other day.

In certain embodiments of the methods and uses of the disclosure as described herein, the administration includes BC-TMM in an amount of about 15 mg. For example, in certain embodiments, the administration includes multiple mini-tablets or a unit dose comprising multiple mini-tablets having a combined amount of BC-TMM of 15 mg.

In certain embodiments of the methods and uses of the disclosure as described herein, the one or more mini-tablets or the unit dose is administered in a fasted state. For example, in certain embodiments, fasted state is following an overnight fast. In certain embodiments, the administration is on an empty stomach, e.g., at least 1 hour before meal or at least 2 hours after meal.

### EXAMPLES

The methods of the disclosure are illustrated further by the following Examples, which is not to be construed as limiting the disclosure in scope or spirit to the specific procedures and compounds described therein.

### Example 1: Preparation of Low Dose Formulations (“Mini-Tablets”)

Various low dose formulations of BC-TTM, referred herein as “mini-tablets” including 1.25 mg of BC-TTM and the excipients as shown in Table 1 and Table 2 were prepared. Upon final blending of BC-TTM and the excipients, the tablet cores were produced using a compression machine according to commonly used methods for the manufacturing of tablet dosage forms, Subsequently, the tablet cores were subject to coating according to common coating methods. Formulation #1, Generations 1, 2 and 3 included the hydrophobic lubricant magnesium stearate (Table 1). For Formulation # 2, Generations 1, 2, 3 and 4, the lubricant was changed to the hydrophilic lubricant sodium stearyl fumarate (Table 2).

TABLE 1
Components of Formulation #1, Generations 1, 2 and 3
FORMULATION #1

| | Generation 1 (F1G1) | | Generation 2 (F1G2) | | Generation 3 (F1G3) | |
|---|---|---|---|---|---|---|
| | Quantity | mg/tablet | Quantity | mg/tablet | Quantity | mg/tablet |
| | (%) | | (%) | | (%) | |
| BC-TTM | 8.33 | 1.250 | 8.33 | 1.250 | 8.33 | 1.250 |
| Sodium Bicarbonate | 24.99 | 3.749 | 24.99 | 3.749 | 24.99 | 3.749 |
| Microcrystalline | 31.84 | 4.776 | 31.84 | 4.776 | 32.84 | 4.926 |
| Cellulose | | | | | | |
| (Avicel PH112) | | | | | | |
| Lactose Monohydrate | 31.84 | 4.776 | 31.84 | 4.776 | 32.84 | 4.926 |
| Fast-Flo 316 | | | | | | |
| Croscarmellose Sodium | 2.00 | 0.300 | 2.00 | 0.300 | n/a | n/a |
| (Ac-Di-Sol) | | | | | | |
| Magnesium | | | | | | |
| Stearate #5712 | 0.50 | 0.075 | 0.50 | 0.075 | 0.50 | 0.075 |
| Sub-total | 99.50 | 14.93 | 99.50 | 14.93 | 99.50 | 14.93 |
| Magnesium | | | | | | |
| Stearate #5712 | 0.50 | 0.075 | 0.50 | 0.075 | 0.50 | 0.075 |
| (Extragranular) | | | | | | |
| Total Core | 100 | 15.000 | 100 | 15.000 | 100 | 15.000 |
| Coating Components | % weight | mg/tablet | % weight | mg/tablet | % weight | mg/tablet |
| | gain | | gain | | gain | |
| Carnauba Wax | n/a | n/a | 1.00 | 0.1500 | 1.00 | 0.1500 |
| Powdered #1 NF | | | | | | |
| Opadry 200 Clear | 8.00 | 1.200 | 15.00 | 2.2500 | 15.00 | 2.2500 |
| 203A190001 | | | | | | |
| Acryl-EZE White | 10.0 | 1.620 | 35.00 | 6.0375 | 35.00 | 6.0375 |
| Total Coated Tablet | 118 | 17.820 | 150 | 23.2875 | 150 | 23.2875 |

TABLE 2
Components of Formulation #2, Generations 1, 2, 3 and 4
FORMULATION #2

| | Generation 1 (F2G1) | | Generation 2 (F2G2) | | Generation 3 (F2G3) | | Generation 4 (F2G4) | |
|---|---|---|---|---|---|---|---|---|
| | Quantity | mg/table | Quantity | mg/table | Quantity | mg/tablet | Quantity | mg/tablet |
| | (%) | | (%) | | (%) | | (%) | |
| BC-TTM | 8.33 | 1.250 | 8.33 | 1.250 | 8.33 | 1.250 | 8.33 | 1.250 |
| Sodium Bicarbonate | 24.99 | 3.749 | 24.99 | 3.749 | 24.99 | 3.749 | 24.99 | 3.749 |
| Microcrystalline | 65.68 | 9.852 | 65.68 | 9.852 | 65.93 | 9.890 | 65.93 | 9.890 |
| Cellulose | | | | | | | | |
| (Avicel PH112) | | | | | | | | |
| Polyplasdone XL | 0.25 | 0.038 | 0.25 | 0.038 | n/a | n/a | n/a | n/a |
| (Crospovidone) | | | | | | | | |
| Sodium Stearyl | | | | | | | | |
| Fumarate | 0.25 | 0.038 | 0.25 | 0.038 | 0.25 | 0.038 | 0.25 | 0.038 |
| Sub-total | 99.50 | 14.93 | 99.50 | 14.93 | 99.50 | 14.93 | 99.50 | 14.93 |
| Sodium Stearyl | 0.50 | 0.075 | 0.50 | 0.075 | 0.50 | 0.075 | 0.50 | 0.075 |
| Fumarate | | | | | | | | |
| (Extragranular) | | | | | | | | |
| Total Core | 100 | 15.000 | 100 | 15.000 | 100 | 15.000 | 100 | 15.000 |
| Coating Components | % weight | mg/tablet | % weight | mg/tablet | % weight | mg/tablet | % weight | mg/tablet |
| | gain | | gain | | gain | | gain | |
| Carnauba Wax | n/a | n/a | 1.00 | 0.1500 | 1.00 | 0.1500 | 1.00 | 0.1500 |
| Powdered #1 NF | | | | | | | | |
| Opadry 200 Clear | 8.00 | 1.200 | 15.00 | 2.2500 | 15.00 | 2.2500 | 20.00 | 3.0300 |
| 203A190001 | | | | | | | | |
| Acryl-EZE White | 10.0 | 1.620 | 35.00 | 6.0375 | 35.00 | 6.0375 | 35.00 | 6.3630 |
| Total Coated Tablet | 118 | 17.820 | 150 | 23.2875 | 150 | 23.2875 | 156 | 24.543 |

### Example 2: Accelerated 4 Week Stability of Mini-Tablets

The objective of the stability study was to assess the stability profile of several of BC-TTM mini-tablet formulations. The stability was evaluated using observation of one tablet (for product appearance) and HPLC/UV (200 to 400 nm) analysis of injection from one tablet sample preparation (for assay of BC-TTM and impurities content). The stability of the mini-tablets was compared to a tablet comprising 5 mg of ALX1840, having a formulation as shown in Table 3.

The stability of the mini-tablets of the disclosure was evaluated at start (“ATST”), at week 1 (“1W”), at week 2 (“2W”), and at week 4 (“4W”) when stored at 5° C., at 25° C. at 60% relative humidity (RH), and 40° C. at 75% RH. Table 4 provides evaluation of Formulation #1, Generation 2 (F1G2) of Example 1; Table 5 provides evaluation of Formulation #2, Generation #2 (F2G2) of Example 1; and Table 6 provides evaluation of 5mg tablet. LTLOQ as used herein means “lower than limit-of-quantification”; ND as used herein means “not determined.” For Tables 4-7 and 10, the reported amounts of TM0 were measured as TM0 in its anion form ([MoO₄]²⁻), whereas the TM0 in the remainder of the disclosure is reported in terms of its choline salt form. The “Total Impurities” amounts reported in Tables 4-7 and 10, therefore, were calculated using the amount of TM0 in its anion form, whereas the “Total Impurities” amounts reported in the remainder of the disclosure were calculated using the amount of TM0 in its choline salt form.

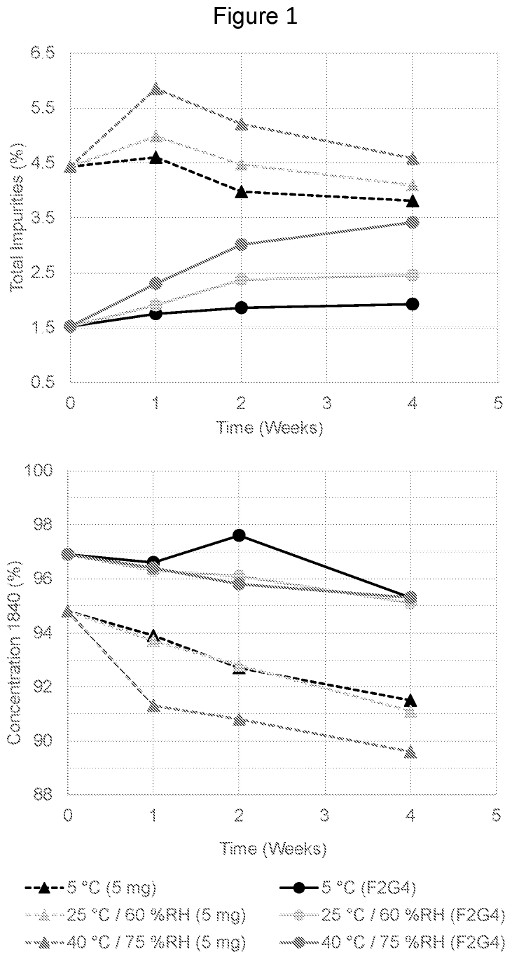

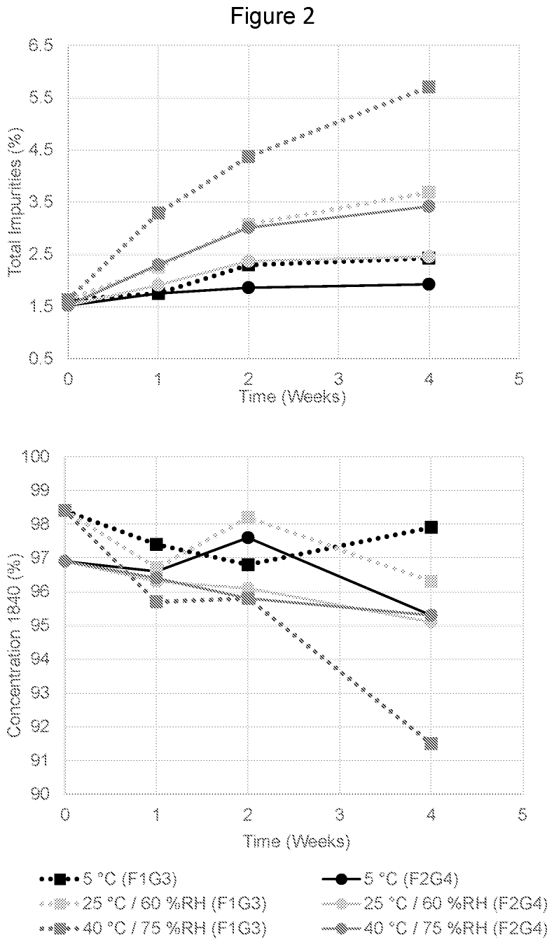

Surprisingly, the 1.25 mg F2G2 mini-tablet showed greater stability compared to the 5 mg tablet as illustrated by the lower concentration of total impurities (%) over time; and the higher concentration of BC-TTM (%) over time (FIGS. 1 and Table 7). In addition, FIG. 2 illustrates that the 1.25 mg F2G2 mini-tablet also showed greater stability compared to the 1.25 mg F1G2 mini-tablet.

TABLE 3
Formulation of 5 mg BC-TTM Tablet

| Component | Amount Per Tablet | |
|---|---|---|
| BC-TTM | 5 | mg |
| Tribasic calcium phosphate | 50.2 | mg |
| Sodium carbonate, anhydrous | 3.0 | mg |
| Sodium starch glycolate | 1.2 | mg |
| Magnesium stearate | 0.6 | mg |
| OPADRY ® Complete Film | 3.6 | mg |
| Coating System 03K19229 Clear | | |
| Acryl-EZE White | 4.4 | mg |

TABLE 4
Stability of F1G2 Mini-Tablet

| | | Storage Condition: | | | | | |
|---|---|---|---|---|---|---|---|
| | | | | 1 W | 1 W | | 2 W |
| | | | 1 W | 25 C./ | 40 C./ | 2 W | 25 C./ |
| Method: | | ATST | 5 C. | 60% RH | 75% RH | 5 C. | 60% RH |
| Product Appearance | | X | X | X | X | X | X |
| (visual) | | | | | | | |
| BC-TTM (% LC) | | 98.4 | 97.4 | 96.7 | 95.7 | 96.8 | 98.2 |
| Individual Specified | | 0.12 | 0.13 | 0.20 | 0.35 | 0.20 | 0.30 |
| Impurity TMO | | | | | | | |
| Individual | TM1 | ND | ND | ND | ND | ND | ND |
| Specified | TM2 | 0.53 | 0.55 | 0.59 | 0.65 | 0.52 | 0.60 |
| Impurities | TM3 | 0.44 | 0.49 | 0.63 | 0.86 | 0.81 | 1.06 |
| | Dimer S6 | 0.05 | 0.06 | 0.15 | 0.48 | 0.06 | 0.16 |
| | Dimer S7 | 0.07 | 0.08 | 0.14 | 0.29 | 0.08 | 0.18 |

| | RRT | Area % | RRT | Area % | RRT | Area % | RRT | Area % | RRT | Area % | RRT | Area % |
|---|---|---|---|---|---|---|---|---|---|---|---|---|
| Individual | 1.67 | LTLOQ | 1.67 | LTLOQ | 1.67 | LTLOQ | 1.63 | LTLOQ | | | | |
| Unspecified | | | | | | | | | | | 1.91 | LTLOQ |
| Impurities | | | 1.92 | LTLOQ | 1.92 | 0.09 | 1.92 | 0.17 | 1.93 | 0.08 | 1.93 | 0.18 |
| | 1.96 | 0.14 | 1.96 | 0.15 | 1.96 | 0.18 | 1.96 | 0.20 | 1.97 | 0.20 | 1.97 | 0.22 |
| | 1.97 | 0.10 | 1.97 | 0.11 | 1.97 | 0.15 | 1.97 | 0.17 | 1.98 | 0.14 | 1.98 | 0.19 |
| | 2.02 | 0.19 | 2.02 | 0.18 | 2.02 | 0.14 | 2.02 | 0.11 | | | | |
| | | | | | | | | | 2.03 | 0.21 | 2.03 | 0.18 |
| Total | 1.63 | | 1.75 | | 2.26 | | 3.29 | | 2.29 | | 3.07 | |
| Impurities | | | | | | | | | | | | |

| | | Storage Condition: | | | |
|---|---|---|---|---|---|
| | | 2 W | | 4 W | 4 W |
| | | 40 C./ | 4 W | 25 C./ | 40 C./ |
| Method: | | 75% RH | 5 C. | 60% RH | 75% RH |
| Product Appearance | | X | X | X | X |
| (visual) | | | | | |
| BC-TTM (% LC) | | 95.8 | 97.9 | 96.3 | 91.5 |
| Individual Specified | | 0.53 | 0.17 | 0.32 | 0.681 |
| Impurity TMO | | | | | |
| Individual | TM1 | ND | ND | ND | ND |
| Specified | TM2 | 0.66 | 0.57 | 0.67 | 0.75 |
| Impurities | TM3 | 1.44 | 0.77 | 1.17 | 1.77 |
| | Dimer S6 | 0.50 | 0.09 | 0.38 | 0.95 |
| | Dimer S7 | 0.25 | 0.19 | 0.37 | 0.39 |

| | RRT | Area % | RRT | Area % | RRT | Area % | RRT | Area % |
|---|---|---|---|---|---|---|---|---|
| Individual | | | | | | | 1.46 | LTLOQ |
| Unspecified | | | | | | | 1.50 | 0.06 |
| Impurities | | | | | | | 1.66 | 0.06 |
| | 1.91 | 0.06 | | | | | 1.91 | 0.08 |
| | 1.93 | 0.33 | | | | | | |
| | 1.97 | 0.24 | | | | | | |
| | 1.98 | 0.20 | 1.98 | 0.10 | 1.98 | 0.24 | 1.98 | 0.35 |
| | | | 2.02 | 0.19 | 2.02 | 0.23 | 2.02 | 0.15 |
| | 2.03 | 0.16 | 20.3 | 0.15 | 2.03 | 0.18 | 2.03 | 0.14 |
| | | | 2.08 | 0.20 | 2.08 | 0.14 | 12.08 | 0.09 |
| | | | | | | | 2.13 | 0.15 |
| | | | | | | | 12.19 | 0.07 |
| Total | 4.37 | | 2.43 | | 3.69 | | 5.7045 | |
| Impurities | | | | | | | | |

X = Round, white coated tablet;
% LC: 93.1, 101.1, 91.8, 102.1, 93.1, 101.0, 91.3, 100.0, 95.1, 102.6;
Content Uniformity (% LC);
Average = 97.1%;
St. deviation = 4.6;
RSD = 4.8%;
Acceptance Value = 12.5%;
Conforms

TABLE 5
Stability of F2G2 Mini-Tablet

| | | Storage Condition: | | | | | |
|---|---|---|---|---|---|---|---|
| | | | | 1 W | 1 W | | 2 W |
| | | | 1 W | 25 C./ | 40 C./ | 2 W | 25 C./ |
| Method: | | ATST | 5 C. | 60% RH | 75% RH | 5 C. | 60% RH |
| Product Appearance | | X | X | X | X | X | X |
| (visual) | | | | | | | |
| BC-TTM (% LC) | | 96.9 | 96.6 | 96.3 | 96.4 | 97.6 | 96.1 |
| Individual Specified | | 0.19 | 0.21 | 0.24 | 0.29 | 0.21 | 0.26 |
| Impurity TMO | | | | | | | |
| Individual | TM1 | ND | ND | ND | ND | ND | ND |
| Specified | TM2 | 0.40 | 0.40 | 0.46 | 0.46 | 0.42 | 0.46 |
| Impurities | TM3 | 0.58 | 0.61 | 0.70 | 0.79 | 0.84 | 0.96 |
| | Dimer S6 | LTLOQ | LTLOQ | 0.06 | 0.14 | LTLOQ | 0.07 |
| | Dimer S7 | 0.06 | 0.07 | 0.08 | 0.13 | ND | 0.10 |

| | RRT | Area % | RRT | Area % | RRT | Area % | RRT | Area % | RRT | Area % | RRT | Area % |
|---|---|---|---|---|---|---|---|---|---|---|---|---|
| Individual | | | | | 1.67 | LTLOQ | | | | | | |
| Unspecified | | | | | 1.92 | 0.05 | 1.92 | 0.08 | | | 1.93 | 0.07 |
| Impurities | 1.96 | 0.11 | 1.96 | 0.12 | 1.96 | 0.13 | 1.96 | 0.18 | 1.97 | 0.14 | 1.97 | 0.17 |
| | 1.97 | 0.06 | 1.97 | 0.07 | 1.97 | 0.08 | 1.97 | 0.11 | 1.98 | 0.10 | 1.98 | 0.12 |
| | 2.02 | 0.12 | 2.02 | 0.12 | 2.02 | 0.11 | 2.02 | 0.12 | | | | |
| | | | | | | | | | 2.03 | 0.16 | 2.03 | 0.16 |
| Total | 1.52 | | 1.75 | | 1.91 | | 2.30 | | 1.86 | | 2.37 | |
| Impurities | | | | | | | | | | | | |

| | | Storage Condition: | | | |
|---|---|---|---|---|---|
| | | 2 W | | 4 W | 4 W |
| | | 40 C./ | 4 W | 25 C./ | 40 C./ |
| Method: | | 75% RH | 5 C. | 60% RH | 75% RH |
| Product Appearance | | X | X | X | X |
| (visual) | | | | | |
| BC-TTM (% LC) | | 95.8 | 95.3 | 95.1 | 95.3 |
| Individual Specified | | 0.32 | 0.18 | 0.23 | 0.32 |
| Impurity TMO | | | | | |
| Individual | TM1 | ND | ND | ND | ND |
| Specified | TM2 | 0.46 | 0.45 | 0.48 | 0.49 |
| Impurities | TM3 | 1.15 | 0.78 | 0.92 | 1.24 |
| | Dimer S6 | 0.16 | LTLOQ | 0.11 | 0.30 |
| | Dimer S7 | 0.18 | 0.14 | 0.22 | 0.32 |

| | RRT | Area % | RRT | Area % | RRT | Area % | RRT | Area % |
|---|---|---|---|---|---|---|---|---|
| Individual | 1.91 | 0.06 | | | | | 1.9 | 0.07 |
| Unspecified | 1.93 | 0.11 | | | | | | |
| Impurities | 1.97 | 0.25 | | | | | | |
| | 1.98 | 0.15 | 1.98 | LTLOQ | 1.98 | 0.08 | 1.97 | 0.15 |
| | | | 2.02 | 0.13 | 2.02 | 0.15 | 2.01 | 0.22 |
| | 2.03 | 0.18 | 2.03 | 0.10 | 2.03 | 0.12 | 2.02 | 0.15 |
| | | | 2.08 | 0.15 | 2.08 | 0.13 | 2.07 | 0.15 |
| Total | 3.01 | | 1.93 | | 2.45 | | 3.42 | |
| Impurities | | | | | | | | |

X = Round, white coated tablet;
% LC: 95.6, 96.1, 100.7, 93.2, 94.9, 95.2, 96.6, 93.8, 94.1, 95.7;
Content Uniformity (% LC):
Average = 95.6%;
St. deviation = 2.1;
RSD = 2.2%;
Acceptance Value = 7.9%;
Conforms

TABLE 6
Stability of 5 mg Tablet

| | | Storage Condition: | | | | | |
|---|---|---|---|---|---|---|---|
| | | | | 1 W | 1 W | | 2 W |
| | | | 1 W | 25 C./ | 40 C./ | 2 W | 25 C./ |
| Method: | | ATST | 5 C. | 60% RH | 75% RH | 5 C. | 60% RH |
| Product Appearance | | Y | Y | Y | Y | Y | Y |
| (visual) | | | | | | | |
| BC-TTM (% LC) | | 94.8 | 93.9 | 93.7 | 91.3 | 92.7 | 92.8 |
| Individual Specified | | 1.52 | 1.66 | 2.02 | 2.44 | 1.56 | 2.02 |
| Impurity TMO | | | | | | | |
| Individual | TM1 | 0.36 | 0.40 | 0.50 | 0.61 | 0.20 | 0.32 |
| Specified | TM2 | 0.80 | 0.84 | 0.85 | 0.87 | 0.62 | 0.65 |
| Impurities | TM3 | 1.27 | 1.31 | 1.30 | 1.30 | 1.12 | 1.16 |
| | Dimer S6 | 0.27 | 0.19 | 0.17 | 0.22 | 0.21 | 0.08 |
| | Dimer S7 | ND | ND | ND | ND | ND | ND |

| | RRT | Area % | RRT | Area % | RRT | Area % | RRT | Area % | RRT | Area % | RRT | Area % |
|---|---|---|---|---|---|---|---|---|---|---|---|---|
| Individual | 0.44 | 0.10 | 0.45 | 0.13 | 0.44 | 0.15 | 0.44 | 0.19 | 0.45 | 0.19 | 0.45 | 0.24 |
| Unspecified | | | | | | | 1.28 | LTLOQ | | | | |
| Impurities | 1.48 | LTLOQ | | | 1.48 | LTLOQ | 1.48 | 0.10 | | | | |
| | | | | | | | 1.59 | LTLOQ | | | | |
| | | | | | | | 1.64 | 0.06 | | | | |
| | 1.75 | 0.11 | 1.75 | 0.07 | 1.75 | LTLOQ | | | 1.76 | 0.07 | | |
| | 2.01 | LTLOQ | | | 2.01 | LTLOQ | 2.01 | 10.06 | | | | |
| Total | 4.43 | | 4.6 | | 4.98 | | 5.85 | | 3.97 | | 4.47 | |
| Impurities | | | | | | | | | | | | |

| | | Storage Condition: | | | |
|---|---|---|---|---|---|
| | | 2 W | | 4 W | 4 W |
| | | 40 C./ | 4 W | 25 C./ | 40 C./ |
| Method: | | 75% RH | 5 C. | 60% RH | 75% RH |
| Product Appearance | | Y | Y | | Y |
| (visual) | | | | | |
| BC-TTM (% LC) | | 90.8 | 91.5 | 91.1 | 89.6 |
| Individual Specified | | 2.58 | 1.64 | 2.28 | 2.94 |
| Impurity TMO | | | | | |
| Individual | TM1 | 0.42 | ND | ND | ND |
| Specified | TM2 | 0.55 | 0.75 | 0.64 | 0.50 |
| Impurities | TM3 | 1.30 | 1.34 | 1.18 | 1.14 |
| | Dimer S6 | ND | 0.08 | ND | ND |
| | Dimer S7 | ND | ND | ND | ND |

| | RRT | Area % | RRT | Area % | RRT | Area % | RRT | Area % |
|---|---|---|---|---|---|---|---|---|
| Individual | 0.45 | 0.30 | NA | ND | NA | ND | NA | ND |
| Unspecified | 1.49 | 0.05 | | | | | | |
| Impurities | | | | | | | | |
| Total | 5.20 | | 3.81 | | 4.09 | | 4.58 | |
| Impurities | | | | | | | | |

Y = Triangular, white coated tablet;
% LC: 94.1, 92.9, 93.6, 94.9, 90.7, 94.1, 93.8, 95.6, 93.9, 93.9;
Content Uniformity (% LC):
Average = 93.7%;
St. deviation = 1.3;
RSD = 1.4%;
Acceptance Value = 7.9%;
Conforms

TABLE 7
Comparison of F2G2 mini-tablet with 5 mg tablet

| | Storage Conditions: | | | | | | | | | |
|---|---|---|---|---|---|---|---|---|---|---|
| | | | 1 W | 1 W | | 2 W | 2 W | | 4 W | 4 W |
| | | 1 W | 25 C./ | 40 C./ | 2 W | 25 C./ | 40 C./ | 4 W | 25 C./ | 40 C./ |
| Method: | ATST | 5 C. | 60% RH | 75% RH | 5 C. | 60% RH | 75% RH | 5 C. | 60% RH | 75% RH |

5 mg tablet

| | | | | | | | | | | |
|---|---|---|---|---|---|---|---|---|---|---|
| Product | Y | Y | Y | Y | Y | Y | Y | Y | Y | Y |
| Appearance | | | | | | | | | | |
| (visual) | | | | | | | | | | |
| BC-TTM (% LC) | 94.8 | 93.9 | 93.7 | 91.3 | 92.7 | 92.8 | 90.8 | 91.5 | 91.1 | 89.6 |
| Total Impurities | 4.43 | 4.6 | 4.98 | 5.85 | 3.97 | 4.47 | 5.20 | 3.81 | 4.09 | 4.58 |

F2G2 mini-tablet

| | | | | | | | | | | |
|---|---|---|---|---|---|---|---|---|---|---|
| Product | X | X | X | X | X | X | X | X | X | X |
| Appearance | | | | | | | | | | |
| (visual) | | | | | | | | | | |
| BC-TTM (% LC) | 96.9 | 96.6 | 96.3 | 96.4 | 97.6 | 96.1 | 95.8 | 95.3 | 95.1 | 95.3 |
| Total Impurities | 1.52 | 1.75 | 1.91 | 2.30 | 1.86 | 2.37 | 3.01 | 1.93 | 2.45 | 3.42 |

X = Round, white coated tablet;
Y = Triangular, white coated tablet

### Example 3: Manufacturing Mini-Tablets

Mini-tablets of the disclosure were prepared on a manufacturing scale. The batch formula for the mini-tablet is provided in Table 8 below. Smaller or larger batches using the components and proportions may be produced. The mini-tablets cores were prepared using a dry-granulation process. In short, upon final blending, mini-tablet cores were produced using a compression machine to match its targeted physical attributes. Subsequently, mini-tablet cores were subject to seal coating, sub-coating, and finally enteric coating. The mini-tablet manufacturing processes used commercially available pharmaceutical processing equipment commonly used for the manufacturing of tablet dosage forms.

TABLE 8
Drug Product Batch Formula

| | Quantity per | | Theoretical |
|---|---|---|---|
| | Mini-tablet | | Batch |
| Formulation Components | Mg/unit | % | Quantity (g) |
| BC-TTM | 1.25 | 8.33 | 416.5¹ |
| Sodium Bicarbonate, USP Grade 1 Powder - | 3.75 | 25 | 312.0 |
| Increment 1 | | | |
| Sodium Bicarbonate, USP Grade 1 Powder - | | | 312.0 |
| Increment 2 | | | |
| Sodium Bicarbonate, USP Grade 1 Powder - | | | 625.5 |
| Increment 3 | | | |
| Microcrystalline Cellulose, NF | 9.89 | 65.93 | 3296.5¹ |
| (Avicel PH-112) | | | |
| Sodium Stearyl Fumarate, NF (Intragranular) | 0.11 | 0.75 | 12.50 |
| Sodium Stearyl Fumarate, NF (Extragranular) | | | 25.0² |
| Total Core | 15 | 100 | 5000 |
| Carnauba Wax, NF Powdered #1 | 0.15 | 1 | 16.67 × 3³ |
| Opadry 200 Clear 203A190001 | 3.03 | 20 | 336.7 × 3³ |
| Acryl-EZE White | 6.36 | 35 | 707.0 × 3³ |

| | | |
|---|---|---|
| Purified Water, USP⁴ | q.s. | q.s |

| | |
|---|---|
| Total Theoretical Batch Size: | 5.0 kg blend/~333,333 |
| | mini-tablets core |

¹Drug substance quantity may be adjusted based on lot specific potency and the difference adjusted with Microcrystalline Cellulose, NF quantity.
²The actual quantity will be adjusted based on the actual yield of the milled granules.
³Coating operations performed in three sub-lots of approximate equal size.
⁴Water amount used for preparation of coating dispersions of Opadry 200 Clear and Acryl-EZE White may subject to adjustment based on the batch size and is not part of the finished product except for the residual amount remaining after drying.

### Description of Manufacturing Process and Process Controls

The manufacturing process consisted of compounding of drug substance and excipients in a dry granulation process. The final blend was then compressed into mini-tablet cores. Coating processes started with a seal coating of the cores with Carnauba Wax. Then the seal coated tablets were subject to sub-coating using Opadry 200 Clear followed by enteric coating with Acryl-EZE White. The major processing steps are pre-roller compaction blending, roller compaction and milling of the ribbons, final blending of bulk granules with extragranular excipient, mini-tablet compression, seal coating, sub coating and enteric coating.

Pre-Roller Compaction Blending: Prior to processing, it was confirmed for each batch that the BC-TTM had been dispensed within two days of the manufacturing start date. Sodium Bicarbonate, USP Grade 1 Powder-Increment 1 was charged into a 15L bin then BC-TTM was added into the same 15 L. bin. The bag containing the residual BC-TTM was rinsed with Sodium Bicarbonate, USP Grade 1 Powder-Increment 2 and then added into the same 15L bin. Then these materials were blended in the 15 L bin for 5 minutes at 10 RPM. Then Sodium Bicarbonate, USP Grade 1 Powder-Increment 3 was charged into the 15 L bin and the materials were blended for 10 minutes at 10 RPM.

The blended components were then discharged into interim containers and then de-lumped using a Quadro Comil equipped with a 032R screen (˜812 microns). The de-lumped materials then were charged back into the same 15 L bin and mixed for an additional 5 minutes at 10 RPM. Microcrystalline Cellulose, NF (Avicel PH-112) was de-lumped by passing it through the same Comil fitted with 032R screen and collected in a clean suitable container. The de-lumped Microcrystalline Cellulose, NF (Avicel PH-112) was charged into the same 15L bin and mixed for 15 minutes at 10 RPM.

An equal volume of blend from the 15L bin was added to the Sodium Stearyl Fumarate, NF (Intragranular) and mixed by inverting the bag for approximately 20 seconds. This mixture was co-screened through a 20 mesh hand screen directly into the 15L. bin and blended for 5 minutes at 10 RPM. The pre-roller compaction blend was then discharged into an interim container and the yield and accountability was calculated.

Roller Compaction and Milling of Ribbons: The pre-compaction blend was roller compacted using the Alexanderwerks WP120 roller compactor equipped with 40 mm upper smooth/lower square rollers and a chiller set at 15° C. The ribbons were milled using the integrated inline mill on the Alexanderwerks WP120 roller compactor fitted with 1.0 mm coarse screen and 0.63 mm fine screen at 95 RPM.

Ribbon and milled granule samples were collected from the beginning, middle and end of roller compaction. Upon completion of roller compaction, the milled granules were collected into an interim container for immediate continuation of processing.

Final Blending: Based on the yield of granules collected from the roller compaction and milling step, the weights of the extragranular component (Sodium Stearyl Fumarate, NF) was adjusted. Initially about 50% of the milled granules were charged into a 15 L bin. An equal volume of the milled granules from the remaining granules was added to the Sodium Stearyl Fumarate, NF (Extragranular) and mixed by inverting the bag for approximately 20 seconds and then hand screened through a 20 mesh screen directly into the bin, and then charged the remaining milled granules were charged into the bin. The mix was blended at 10 RPM for 5 minutes.

Final blend uniformity samples were collected from ten (10) locations in triplicate from the bin using a disposable 0.5 ml sample thief. An approximately 100 g sample from the bin was also collected and then the final blend was discharged into a foil bag, double lined with polyethylene bags with one desiccant in the headspace of the outer polyethylene bag. Air was removed from the polyethylene bags prior to closure with zip ties. Similarly, air was removed from the foil bag and then purged with nitrogen for approximately 3 minutes prior to heat sealing. The yield and accountability were calculated. The foil bag was then placed into a foil-lined fiber drum and returned to 2-8° C. storage.

Mini-tablet Core: The BC-TTM Final Blend (8.33% by weight based on the weight of the core) was compressed into mini-tablets cores using a Korsch XL 100 Pro Tablet Press equipped with 3 mm Round Multi Tip tooling and the force feeder. The compressed tablets were dedusted using a Key tablet deduster and metal checked using a Lock Met30+ Metal Detector. The mini-tablets were compressed to a target weight of 15 mg/unit and complying with other physical attributes. In-process samples were collected and tested for physical attributes at predetermined time interval during compression to ensure product quality.

Bulk core tablets were collected into a foil bag, double lined with polyethylene bags with one desiccant in the headspace of the outer polyethylene bag. As much air as possible was removed from the polyethylene bags prior to closure with zip ties. The foil bag also went through the process to remove air as much as possible and then it was purged with nitrogen for approximately 3 minutes prior to heat sealing. The foil bag was then placed into a foil-lined fiber drum and returned to 2-8° C. storage.

Enteric Coating of Mini-tablets: Three sub-lots, with almost equal pan load size and identical coating process, are required to coat the whole theoretical batch.

A seal-coat coating of Carnauba Wax, NF Powdered #1 was applied on to the mini-tablets cores using a pan-coating system. Core mini-tablets were seal coated in a Compu-Lab coater fitted with a 15″ pan to a theoretical weight gain of 1%.

A sub-coat coating dispersion was prepared at 20% solid content using Opadry 200 Clear (203A190001) coating system and purified water. Core tablets were sub coated in a Compu-Lab coater fitted with 15″ pan to a theoretical weight gain of 20%±1%,

An enteric coat coating dispersion was prepared at 20% solid content using Acryl-EZE White coating system and purified water. Sub coated tablets were coated in a Compu-Lab coater fitted with 15″ pan to a theoretical weight gain of 35%±1%.

Upon coating completion, the bulk enteric-coated tablets were collected in a foil bag, double lined with polyethylene bags with one desiccant in the headspace of the outer polyethylene bag. As much air as possible was removed from the polyethylene bags prior to closure with zip ties. The foil bag also had as much air as possible removed and then it was purged with nitrogen for approximately 3 minutes prior to heat sealing. The foil bag was then placed into a foil-lined fiber drum and returned.

A summary of the drug product manufacturing in-process controls is provided in Table 9.

TABLE 9
Drug Product Manufacturing In-Process Controls

| Process Step | Test | Method | Limits for Mini-tablets |
|---|---|---|---|
| Compressing as | Weight | Weight check | Target weight 15 mg/Mini-tablet Core |
| Mini-tablets | | composite sample of | (Composite weight variation NMT ± 5%) |
| Core | | 10 tablets | |
| | Hardness | Hardness Tester | Report results (Target - |
| | | | 1.0 kp-2.0 kp approx.) |
| | Friability | Friability Tester | <1.0% weight loss |
| | Thickness | Caliper | Report results (Target 2.0 mm) |
| Seal Coating | Weight | Weight check | 1% |
| | gain | composite sample of | |
| | | 20 or more tablets | |
| Sub Coating | Weight | Weight check | 20% (±1%) |
| | gain | composite sample of | |
| | | 20 or more tablets | |
| Enteric Coating | Weight | Weight check | 35% (±1%) |
| | gain | composite sample of | |
| | | 20 or more tablets | |

There were no significant differences between 1.25 mg mini-tablet formulations produced by different batches (Table 10).

TABLE 10
Comparison Between Batch 1 and Batch 2 Mini-Tablet Samples

| Product | Batch 1 | Batch 2 |
|---|---|---|
| Presentation | 1.25 mg Mini-tablet | 1.25 mg Mini-tablet |
| BC-TTM | 97.2 | 100.3 |
| TM0 | 0.075 | LTLOQ |
| TM1 | ND | ND |
| TM2 | 0.15 | 0.12 |
| TM3 | 0.24 | 0.22 |
| Dimer S6 | LTLOQ | LTLOQ |
| Dimer S7 | ND | ND |
| Total Impurities | 0.57 | 0.52 |

### Example 4: Six-Month Stability of Capsules Comprising Low Dose Formulation

Mini-tablets prepared according to Example 3 were placed in hydroxypropyl methylcellulose (HPMC) sprinkle capsules. Each HPMC capsule contained four (4) individual 1.25 mg mini-tablets, The capsules were stored in 60 cc HDPE WM round bottle ((00601-11-01) (33/400) Q024847) closed with DPC CRH11100 33MM WHT SECURX RIBD SIDE PP CRC TXT (7821H1-G1 263131). Each bottle contained 30 capsules.

The stability of the capsules was evaluated based on product appearance, assay/impurities, dissolution, and moisture when stored at 5° C. and at 25° C./60% RH. The stability data measured at 0, 1, 2, 3, 4, 5, 6, and 12 months is provided in Tables 11 and 12 for samples stored at 5° C. and 25° C./60% RH conditions, respectively. LTLOQ as used herein means “lower than limit-of-quantification”; ND as used herein means “not determined.”

TABLE 11
Stability Data for Capsules Stored at 5° C.

| Storage | Time | 1 | 2 | 3 | 4 | 6 | 12 |
|---|---|---|---|---|---|---|---|
| Condition: | Zero | Month | Month | Month | Month | Month | Month |
| Product | White | White | White | Conforms | Conforms | Conforms | Conforms |
| Appearance | capsule | capsule | capsule | | | | |
| (visual) | containing | containing | containing | | | | |
| | 4 white | 4 white | 4 white | | | | |
| | coated | coated | coated | | | | |
| | white mini- | white mini- | white mini- | | | | |
| | tablets | tablets | tablets | | | | |
| BC-TTM | 100.3 | 98.6 | 99.4 | 99.1 | 96.3 | 99.3 | 98.9 |
| Individual | LTLOQ | 0.22 | 0.20 | 0.33 | 0.32 | 0.43 | 0.36 |
| Specified | | | | | | | |
| Impurity | | | | | | | |
| TMO | | | | | | | |
| Individual | TM1 = ND | TM1 = ND | TM1 = ND | TM1 = ND | TM1 = ND | TM1 = ND | TM1 = ND |
| Specified | TM2 = 0.12 | TM2 = 0.15 | TM2 = 0.15 | TM2 = 0.15 | TM2 = 0.15 | TM2 = 0.16 | TM2 = 0.18 |
| Impurities | TM3 = 0.22 | TM3 = 0.20 | TM3 = 0.19 | TM3 = 0.22 | TM3 = 0.20 | TM3 = 0.21 | TM3 = 0.22 |
| | Dimer | Dimer | Dimer | Dimer | Dimer | Dimer | Dimer |
| | S6 = LTLOQ | S6 = 0.09 | S6 = 0.11 | S6 = 0.14 | S6 = 0.15 | S6 = 0.19 | S6 = 0.20 |
| | Dimer | Dimer | Dimer | Dimer | Dimer | Dimer | Dimer |
| | S7 = ND | S7 = 0.13 | S7 = 0.14 | S7 = 0.14 | S7 = 0.17 | S7 = 0.19 | S7 = 0.18 |
| Individual | 0.18 | 0.17 | 0.17 | 0.10 | 0.14 | 0.14 | 0.21 |
| Unspecified | | | | | | | |
| Impurities | | | | | | | |
| Total Impurities | 0.52 | 0.96 | 0.95 | 1.07 | 1.14 | 1.32 | 1.35 |

TABLE 12
Stability Data for 1.25 mg Mini-Tablets Stored at 25° C./60% RH

| Storage | Time | 1 | 2 | 3 | 4 | 6 | 12 |
|---|---|---|---|---|---|---|---|
| Condition: | Zero | Month | Month | Month | Month | Month | Month |
| Product | White | White | White | Conforms | Conforms | Conforms | Conforms |
| Appearance | capsule | capsule | capsule | | | | |
| (visual) | containing 4 | containing 4 | containing 4 | | | | |
| | white coated | white coated | white coated | | | | |
| | white mini- | white mini- | white mini- | | | | |
| | tablets | tablets | tablets | | | | |
| BC-TTM | 100.3 | 99.4 | 98.4 | 96.4 | 97.2 | 96.5 | 94.4 |
| Individual | LTLOQ | 0.32 | 0.33 | 0.55 | 0.58 | 0.76 | 1.09 |
| Specified | | | | | | | |
| Impurity | | | | | | | |
| TMO | | | | | | | |
| Individual | TM1 = ND | TM1 = ND | TM1 = ND | TM1 = ND | TM1 = ND | TM1 = ND | TM1 = ND |
| Specified | TM2 = 0.12 | TM2 = 0.15 | TM2 = 0.15 | TM2 = 0.15 | TM2 = 0.16 | TM2 = 0.17 | TM2 = 0.19 |
| Impurities | TM3 = 0.22 | TM3 = 0.22 | TM3 = 0.23 | TM3 = 0.30 | TM3 = 0.27 | TM3 = 0.32 | TM3 = 0.43 |
| | Dimer | Dimer | Dimer | Dimer | Dimer | Dimer | Dimer |
| | S6 = LTLOQ | S6 = 0.20 | S6 = 0.25 | S6 = 0.36 | S6 = 0.36 | S6 = 0.47 | S6 = 0.69 |
| | Dimer | Dimer | Dimer | Dimer | Dimer | Dimer | Dimer |
| | S7 = ND | S7 = 0.25 | S7 = 0.27 | S7 = 0.26 | S7 = 0.31 | S7 = 0.33 | S7 = 0.30 |
| Individual | 0.18 | 0.34 | 0.22 | 0.09 | 0.14 | 0.4 | 0.83 |
| Unspecified | | | | | | | |
| Impurities | | | | | | | |
| Total Impurities | 0.52 | 1.47 | 1.45 | 1.71 | 1.83 | 2.44 | 3.54 |

Another set of capsules (4 mini-tablets per capsule, prepared and stored as noted above, except that the bottles were closed with 33mm SCRX RI BD SIDE WHT PP CRC TXT TOP (HS130-35 7903HI-1CI 263455)) containing another batch of 1,25 mg mini-tablets prepared according to Example 3 (a so-called “second batch”) were also tested for long-term stability, relative to the standards provided in Table 14. Table 13 provides the results of the stability evaluation at 3 months of storage at 5° C. and at 25° C./60% RH; 6 months of storage at 25160% RH: and 12 months of storage at 5° C.

TABLE 13
3, 6, and 12-Month Stability Data for Second Batch of 1.25 mg Mini-Tablets

| Product | 3 Months, | 3 Months, 25° | 6 Months, 25° | 12 Months, |
|---|---|---|---|---|
| Presentation | 5° C. | C./60% RH | C./60% RH | 5° C. |
| Product | Conforms | Conforms | Conforms | Conforms |
| Appearance | | | | |
| (visual) | | | | |
| BC-TTM | 100, 99.7 | 97.5, 98.3 | 97.0, 96.8 | 99.5, 100.9 |
| | Avg = 100.1 | Avg = 97.9 | Avg = 96.9 | Avg = 100.2 |
| TM0 | 0.3, 0.3 | 0.3, 0.2 | 0.7, 0.8 | 0.2, 0.2 |
| | Avg = 0.3 | Avg = 0.3 | Avg = 0.8 | Avg = 0.2 |
| TM1 | ND | ND | ND | ND |
| TM2 | 0.13, 0.13 | 0.19, 0.14 | 0.2, 0.2 | 0.1, 0.1 |
| TM3 | 0.20, 0.20 | 0.30, 0.27 | 0.4, 0.4 | 0.3, 0.3 |
| Dimer S6 | 0.17, 0.16 | 0.37, 0.36 | 0.5, 0.5 | 0.2, 0.2 |
| Dimer S7 | 0.19, 0.18 | 0.23, 0.27 | 0.3, 0.3 | 0.2, 0.2 |
| Individual | 0.12; | 0.11, 0.11; | 0.8, 0.8 | 0.1, 0.1; |
| Unspecified | Avg = 0.13 | Avg = 0.11 | Avg = 0.8 | Avg = 0.1 |
| Impurities | | | | |
| Total Impurities | 1.1, 1.1; | 1.9, 1.7; | 2.6, 2.7 | 1.1, 1.0 |
| | Avg = 1.1 | Avg = 1.8 | Avg = 2.7 | Avg = 1.1 |

TABLE 14
Stability Testing Standards

| | |
|---|---|
| Test | Standard |

Physical

| | |
|---|---|
| Appearance Packaging | White capsule containing four white |
| | to off-white mini-tablets |

Chemical

| | |
|---|---|
| Identification by HPLC/UV | The UV spectrum of the sample (200 to |
| | 400 nm) conforms to that of the reference |
| | standard |
| Identification by | Difference between sample and standard |
| HPLC Retention Time | retention time is NMT 2.0% |
| Assay | 90.0-110.0% label claim |
| Impurities by HPLC | TM0: ≤3.0% |
| Impurities by HPLC | TM1: ≤0.5% TM2: ≤1.0% TM3: ≤3.0% |
| | Dimer S6: ≤1.0% Dimer S7: ≤1.0% |
| | Unknown Impurities: Any other |
| | impurity ≤0.5% |
| Content uniformity | Total impurities: ≤6.0% |

### Example 5: Relative Bioavailability of Two Oral Formulations of BC-TTM in Healthy Adult Participants

A phase 1, randomized, 2-period, 2-sequence, crossover with parallel-group extension, open -label study was conducted to compare the relative bioavailability of 2 oral formulations of BC-TTM in healthy adult participants. The purpose of this study was to assess relative bioavailability of the 1.25 mg enteric-coated (EC) mini-tablet formulation of BC-TTM compared with a 15 mg EC tablet of BC-TTM to assess dose proportionality between 2.5 mg (2×1.25 mg), 5 mg (4×1.25 mg), 10 mg (8×1.25 mg), 15 mg (12×1,25 mg), and 30 mg (24×1.25 mg) EC mini-tablet doses. The 15 mg EC tablet of BC-TTM used in the study had a formulation consisting of the components listed in Table 3. The 1.25 mg EC mini-tablets of BC-TTM were prepared in accordance with Example 3 and the drug product batch formula of Table 8.

This was a 2-period, 2-sequence crossover study with parallel group extension designed to assess the relative bioavailability of equal doses of BC-TTM administered as 1.25 mg EC mini-tablets versus a single 15 mg EC tablet, and to assess dose-proportionality between 2.5 mg (2×1.25 mg), 5 mg (4×1.25 mg), 10 mg (8×1.25 mg), 15 mg (12×1.25 mg), and 30 mg (24×1.25 mg) EC mini-tablet doses in the Dose-Proportionality Extension Period. The safety and tolerability of the 2 formulations of BC-TTM in healthy participants was also assessed. BC-TTM pharmacokinetics (PK) in plasma as measured via total molybdenum (Mo) and plasma ultrafiltrate (PUF) Mo was determined.

TABLE 15
Objectives and Endpoints of Study

| Objective | Endpoints/Estimands | Results |
|---|---|---|
| Primary | | |
| To assess the relative | PK parameters for plasma | Plasma total and PUF molybdenum as surrogate |
| bioavailability of equal doses | total Mo and PUF Mo (Cₘₐₓ, | measures for BC-TTM PK profiles and PK |
| of BC-TTM administered as | AUCₜ, and AUC_{∞}) | parameters were comparable between a single |
| 1.25 mg EC mini-tablets | | dose of BC-TTM administered as 12 × 1.25 mg |
| versus a single 15 mg EC | | EC mini tablets (15 mg total dose) and as |
| tablet | | 1 × 15 mg EC tablet and there were no |
| | | clinically relevant differences between the |
| | | 2 treatment formulations under fasting |
| | | conditions in healthy participants as the |
| | | 90% CIs for Cₘₐₓ, AUCₜ and AUC_{∞} of total |
| | | molybdenum were contained within the 80% |
| | | to 125% bioequivalence limits. |
| Secondary | | |
| To assess dose- | Dose-normalized PK | Plasma total molybdenum PK parameters |
| proportionality between 2.5 | parameters for plasma total | generally showed a dose proportional |
| mg (2 × 1.25 mg), 5 mg (4 × | Mo and PUF Mo (Cₘₐₓ_{_{—}}ₙ, | increase from 2.5 mg to 30 mg for the |
| 1.25 mg), 10 mg (8 × 1.25 | AUCₜ_{_{—}}ₙ, and AUC_{∞}_{_{—}}ₙ) | BC-TTM EC mini-tablet formulation. |
| mg), 15 mg (12 × 1.25 mg), | | Plasma PUF molybdenum PK parameters |
| and 30 mg (24 × 1.25 mg) | | showed a less than dose proportional increase |
| EC mini-tablet doses | | from 2.5 mg to 30 mg for the BC-TTM EC |
| | | mini-tablet formulation. |
| Safety | | |
| To assess the overall safety | Incidence of TEAEs and | No deaths or TESAEs were reported. |
| and tolerability of BC-TTM, | TESAEs, physical | Two participants were discontinued from |
| administered as 1.25 mg EC | examination, vital signs | the study due to increased ALT blood |
| mini-tablets and as a single | measurements, clinical | concentrations following Treatment B. |
| 15 mg EC tablet | laboratory, and 12-lead | All TEAEs were Grade 1 or 2 in severity, |
| | ECG results | except for 2 events of increased blood |
| | | creatine phosphokinase blood concentrations |
| | | of Grade 4 severity reported by 2 (4.3%) |
| | | participants following Treatment B during |
| | | the Two-way Crossover Period. |
| | | The incidence of TEAEs was similar between |
| | | Treatment A (BC-TTM 12 × 1.25 mg EC mini- |
| | | tablets) and Treatment B (BC-TTM single 15 |
| | | mg EC reference tablet), and no dose- |
| | | relationship was observed for the Treatments |
| | | C to F (2.5 mg to 30 mg BC-TTM administered |
| | | as 1.25 mg EC mini-tablets). |
| | | Most commonly reported study intervention- |
| | | related TEAEs were ALT increased, headache, |
| | | and rash. |
| Exploratory | | |
| To explore relationships | CL/F, body weight, and BMI | There was no apparent effect of body |
| between total Mo and PUF | | weight or BMI on BC-TTM PK for any of |
| Mo clearance and body size - | | the treatments evaluated. |
| body weight (kg) and BMI | | |
| (kg/m²) | | |
| To explore PD of BC-TTM | Absolute and percent | There were no apparent differences in BC-TTM |
| either as a single 15 mg EC | changes from pre-dose | PD parameters (plasma total and PUF copper |
| tablet or EC mini-tablets of | baseline of plasma Cu | concentrations) between 12 × 1.25 mg EC |
| 1.25 mg at different total | concentrations: total | mini-tablets and the 15 mg reference EC |
| dose strengths | Cu and PUF Cu | tablet. In the single dose range of 2.5 |
| | | mg to 30 mg, there were modest, transient, |
| | | and dose-dependent mean percentage increases |
| | | from baseline in the maximum plasma total |
| | | copper concentration, most apparent at 8 |
| | | hours post-dose. There were no apparent |
| | | dose-dependent differences in PUF copper |
| | | concentration. |

The study had a Screening Period (Days −28 to −2), the Two-way Crossover Period, consisting of 2 dosing periods (Day 1 to Day 11 each), and a Dose-Proportionality Extension Period. After completing the Screening Period, enrolled participants were admitted to the clinical research unit (CRU) on Day −1 for dosing on Day 1 in Dosing Period 1. If discharged after Dosing Period 1, participants were readmitted to the CRU for Dosing Period 2 following a minimum washout of 14 days after the previous dose, and again for the Dose-Proportionality Extension Period after a minimum washout of 14 days, The end of study (EOS) visit took place 14 days (±2 days) after the dose of BC-TTM in the Dose-Proportionality Extension Period.

The Two-way Crossover Period was a randomized, open-label, 2-way (2-period, 2-sequence), crossover design to assess the relative bioavailability of 12×1.25 mg EC mini-tablets compared with the 15 mg EC tablet currently used in clinical studies. Participants were randomized to one of the two treatments sequences. Randomized treatment assignment were based on Baseline body mass index (BMI), Two strata for EMI (<25, 25 to <32 kg/m²) were used:
Treatment A: BC-TTM 12×1.25 mg EC mini-tablets
Treatment B: BC-TTM single 15 mg EC tablet (reference tablet, currently being tested in the Phase 3 Study WTX101-301)

| | Treatment Sequence | | |
|---|---|---|---|
| Sequence Number | Period 1 | Period 2 | Total |
| 1 | A | B | 24 |
| 2 | B | A | 24 |

| | |
|---|---|
| Total | 48 |

Blood samples for PK analysis of total and PUF Mo (as surrogate measures of BC-TTM PK) and pharmacodynamic (PD)/biomarkers were collected in each dosing period on Day 1 at pre-dose, and postdose at 1, 2, 3, 4, 5, 6, 8, 12 and 24 hours (Day 2) and then at 24 hour intervals on Days 3, 4, 5 6, 7, 8, 9, 10, and 11.

The 336-hour sample for Dosing Period 1 were collected predose in Dosing Period 2. Participants could have been discharged on Day 11 of each dosing period after completion of all procedures and review of all safety data. The end of Dosing Period 2 occurred on Day 15±2 of Dosing Period 2, with the collection of the 336-hour PK sample for Dosing Period 2.

The Dose-Proportionality Extension Period was a re-randomized, open-label, parallel group design to assess the dose-proportionality between 2.5 mg (2×1.25 mg), 5 mg (4×1.25 mg), 10 mg (8×1.25 mg), and 30 mg (24×1.25 mg) EC mini-tablet doses. The 15 mg (12×1.25 mg) dose was not repeated during the Dose-Proportionality Extension Period.

The Dose-Proportionality Extension Period was conducted following completion of the Two-way Crossover Period of the study and after an at least 14-day washout period. Participants were re-randomized as follows:
Treatment C (N=10-12): BC-TTM 2.5 mg (2×1.25 mg EC mini-tablets)
Treatment D (N=10-12): BC-TTM 5 mg (4×1.25 mg EC mini-tablets)
Treatment E (N=10-12): BC-TTM 10 mg (8×1.25 mg EC mini-tablets)
Treatment F (N=10-12): BC-TTM 30 mg (24×1.25 ma EC mini-tablets)

The dose-proportionality evaluation included data obtained from Treatment A of the Two-way Crossover Period (12×1.25 mg EC mini-tablets) to represent a dose of 15 mg,

Re-randomized treatment assignment were based on Baseline body mass index (BMI). Two strata for Baseline BM (<25, 25 to <32 kg/m²) were used. Block randomization was used to equally randomly assign participants to each treatment.

Participants could have been discharged on Day 11 of the Dose-Proportionality Extension Period after completion of all procedures and review of all safety data.

Participants could have been asked or required to stay in the CRU during the Two-way Crossover Period, and/or at the end of the Dose-Proportionality Extension Period before the end of study (EOS) visit, for their own safety, and also to maintain the integrity of the conduct of the study.

The final data showed that of the 48 randomized participants, 44 participants completed the Two-way Crossover Period and 40 participants completed the Dose-Proportionality Extension Period. All 48 (100%) participants randomized in the Two-way Crossover Period were included in the Safety, PKDS-CO, and Full Analysis sets, and all 41 (100%) participants randomized in the Dose-Proportionality Extension Period were included in the Safety, PKDS-E, and Full Analysis sets.

Plasma total and PUF molybdenum as surrogate measures for BC-TTM PK profiles and PK parameters were comparable between a single dose of BC-TTM administered as 12×1.25 mg EC mini tablets (15 mg total dose) and as 1×15 mg EC tablet. There were no clinically relevant differences between the 2 treatment formulations under fasting conditions in healthy participants as the 90% CIs for Cₘₐₓ, AUCₜ and AUC_{∞} of total molybdenum were contained within the 80% to 125% bioequivalence limits.

TABLE 16
Summary of PK Parameters of Plasma Total and
PUF Molybdenum - Two-way Crossover Period

| | | Treatment A | |
|---|---|---|---|
| | | BC-TTM | Treatment B |
| | | 12 × 1.25 mg | BC-TTM |
| | | EC mini-tablets | 1 × 15 mg |
| | | (N = 46) | EC tablet |
| Analyte | PK Parameters^{a} | Arithmetic Mean ± SD (% CV) | (N = 46) |
| Total | t_{lag} (h)^{b} | 0 | 0 |
| | | (0, 3) | (0, 3) |
| molybdenum | tₘₐₓ (h)^{b} | 6.0 | 5.0 |
| | | (3.0, 8.0) | (1.0, 335.8) |
| | Cₘₐₓ (ng/mL) | 243.6 ± 72.5 | 248.5 ± 73.4 |
| | | (29.8) | (29.6) |
| | Cₘₐₓ_{_{—}}ₙ (ng/mL)/(mg) | 73.2 ± 21.8 | 74.7 ± 22.1 |
| | | (29.8) | (29.6) |
| | t1/2 (h) | 126.4 ± 43.7 | 130.4 ± 55.2 |
| | | (34.5) | (42.4) |
| | AUCₜ (h*ng/mL) | 9316.1 ± 3014.3 | 10252.2 ± 6156.8 |
| | | (32.4) | (60.1) |
| | AUCₜ_{_{—}}ₙ (h*ng/mL)/(mg) | 2800.2 ± 906.0 | 3081.5 ± 1850.5 |
| | | (32.4) | (60.0) |
| | AUC_{∞} (h*ng/mL) | 10542.5 ± 2895.0 | 10341.5 ± 2634.7 |
| | | (27.5) | (25.5) |
| | AUC_{∞}_{_{—}}ₙ (h*ng/mL)/(mg) | 3168.8 ± 870.1 | 3108.4 ± 791.9 |
| | | (27.5) | (25.5) |
| | λ_{z} (1/h) | 0.006 ± 0.002 | 0.006 ± 0.002 |
| | | (34.0) | (32.8) |
| | CL/F (L/h)^{c} | 0.3 ± 0.1 | 0.3 ± 0.1 |
| | | (31.2) | (21.0) |
| | V_{d}/F (L)^{c} | 61.4 ± 24.8 | 63.3 ± 27.9 |
| | | (40.3) | (44.0) |
| PUF | tₘₐₓ (h)^{b} | 6.0 | 5.0 |
| | | (3.0, 144.1) | (1.0, 120.0) |
| molybdenum | Cₘₐₓ (ng/mL) | 11.1 ± 4.2 | 11.6 ± 6.2 |
| | | (37.9) | (53.5) |
| | Cₘₐₓ_{_{—}}ₙ (ng/mL)/(mg) | 3.3 ± 1.3 | 3.5 ± 1.9 |
| | | (37.9) | (53.5) |
| | AUCₜ (h*ng/mL) | 401.8 ± 193.7 | 388.9 ± 189.5 |
| | | (48.2) | (48.7) |
| | AUCₜ_{_{—}}ₙ (h*ng/mL)/(mg) | 120.8 ± 58.2 | 116.9 ± 57.0 |
| | | (48.2) | (48.7) |

^{a}PK parameters were calculated based on corrected concentrations and all parameter values (except for λ_{z}) are rounded to one digit after decimal point from source data
^{b}Data presented as mean ± SD (% CV) except for tₘₐₓ and t_{lag} as median (range).
^{c}Molybdenum dose was used to calculate CL/F or V_{d}/F values.

Plasma total molybdenum PK parameters generally showed a dose-proportional increase from 2.5 mg to 30 mg for the BC-TTM EC mini-tablet formulation. Plasma PUF molybdenum PK parameters showed a less than dose-proportional increase from 2.5 mg to 30 mg for the BC-TTM EC mini-tablet formulation. BC-TTM PK were apparently not affected by body weight or BMI.

TABLE 17
Summary of PK Parameters of Plasma Total and PUF Molybdenum - Dose-Proportionality Extension Period

| | | | | | |
|---|---|---|---|---|---|
| | Treatment C | Treatment D | Treatment E | Treatment A | Treatment F |
| | 2.5 mg BC-TTM | 5 mg BC-TTM | 10 mg BC-TTM | 15 mg BC-TTM | 30 mg BC-TTM |
| | (2 × 1.25 mg) | (4 × 1.25 mg) | (8 × 1.25 mg) | (12 × 1.25 mg) | (24 × 1.25 mg) |
| PK | EC mini-tablets | EC mini-tablets | EC mini-tablets | EC mini-tablets | EC mini-tablets |
| Parameters^{a} | (N = 10) | (N = 11) | (N = 9) | (N = 46) | (N = 11) |

Total molybdenum

| | | | | | |
|---|---|---|---|---|---|
| t_{lag} (h)^{b} | 0 | 0 | 0 | 0 | 0 |
| | (0, 3) | (0, 0) | (0, 0) | (0, 3) | (0, 0) |
| tₘₐₓ (h)^{b} | 3.0 | 5.0 | 4.0 | 6.0 | 5.1 |
| | (2.0, 6.0) | (3.0, 6.0) | (3.0, 8.0) | (3.0, 8.0) | (4.0, 8.0) |

Arithmetic Mean ± SD (% CV)

| | | | | | |
|---|---|---|---|---|---|
| Cₘₐₓ (ng/mL) | 41.0 ± 19.5 | 104.4 ± 52.6 | 199.5 ± 55.2 | 243.6 ± 72.5 | 396.0 ± 190.2 |
| | (47.5) | (50.3) | (27.7) | (29.8) | (48.0) |
| t1/2 (h) | NA | 197.7 ± 96.4 | 191.8 ± 79.6 | 126.4 ± 43.7 | 114.7 ± 29.3 |
| | | (48.8) | (41.5) | (34.5) | (25.5) |
| AUCₜ (h*ng/mL) | 1677.9 ± 736.4 | 4053.6 ± 1601.4 | 7439.1 ± 2213 | 9316.1 ± 3014.3 | 16778.2 ± 4707.8 |
| | (43.9) | (39.5) | (29.7) | (32.4) | (28.1) |
| AUC_{∞} (h*ng/mL) | NA | 4920.5 ± 1035.2 | 9057.1 ± 1345.9 | 10542.5 ± 2895.0 | 17842.2 ± 5496.9 |
| | | (21.0) | (14.9) | (27.5) | (30.8) |
| λ_{z} (1/h) | NA | 0.004 ± 0.001 | 0.005 ± 0.003 | 0.006 ± 0.002 | 0.007 ± 0.002 |
| | | (25.6) | (58.1) | (34.0) | (27.6) |
| CL/F (L/h)^{c} | NA | 0.2 ± 0 | 0.2 ± 0 | 0.3 ± 0.1 | 0.4 ± 0.1 |
| | | (20.2) | (14.6) | (31.2) | (27.8) |
| V_{d}/F (L)^{c} | NA | 63.6 ± 21.2 | 68.9 ± 31.4 | 61.4 ± 24.8 | 64.6 ± 18.9 |
| | | (33.3) | (45.6) | (40.3) | (29.3) |

PUF molybdenum

| | | | | | |
|---|---|---|---|---|---|
| tₘₐₓ (h)^{b} | 5.0 | 5.0 | 6.0 | 6.0 | 6.0 |
| | (1.0, 192.0) | (2.0, 340.8) | (2.0, 144.0) | (3.0, 144.1) | (4.0, 8.0) |
| Cₘₐₓ (ng/mL) | 7.2 ± 9.6 | 7.5 ± 12.5 | 15.1 ± 21.0 | 11.1 ± 4.2 | 24.5 ± 12.5 |
| | (131.9) | (167.0) | (138.6) | (37.9) | (44.7) |
| AUCₜ (h*ng/mL) | 384.0 ± 453.5 | 560.5 ± 1169.1 | 474.5 ± 446.7 | 401.8 ± 193.7 | 550.1 ± 114.6 |
| | (118.1) | (208.6) | (94.1) | (48.2) | (20.8) |

^{a}PK parameters were calculated based on corrected concentrations and all parameter values (except for λ_{z}) are rounded to one digit after decimal point.
^{b}Data presented as mean ± SD (% CV) except for tₘₐₓ and t_{lag} as median (range).
^{c}Molybdenum dose was used to calculate CL/F or V_{d}/F values.

Dose-normalized plasma total molybdenum Cₘₐₓ and AUCₜ values decreased moderately with increasing dose, with a decrease more prominent in AUC_{∞} values. For PUF molybdenum, dose-normalized plasma exposure values decreased with increasing dose, indicating that BC-TTM PUF molybdenum exposure increased in a less than dose proportional manner within the BC-TTM dose range of 2.5 mg to 30 mg for the EC mini-tablet formulation (Table 18).

TABLE 18
Summary of Dose-normalized PK Parameters of Plasma Total and PUF Molybdenum - Dose-
Proportionality Extension Period (PKDS-E Set and Treatment A from PKDS-CO Set)

| | | | | | |
|---|---|---|---|---|---|
| | Treatment C | Treatment D | Treatment E | Treatment A | Treatment F |
| | 2.5 mg BC-TTM | 5.0 mg BC-TTM | 10 mg BC-TTM | 15 mg BC-TTM | 30 mg BC-TTM |
| | (2 × 1.25 mg) | (4 × 1.25 mg) | (8 × 1.25 mg) | (12 × 1.25 mg) | (24 × 1.25 mg) |
| | EC mini-tablets | EC mini-tablets | EC mini-tablets | EC mini-tablets | EC mini-tablets |
| PK | (N = 10) | (N = 11) | (N = 9) | (N = 46) | (N = 11) |

| | |
|---|---|
| Parameters^{a} | Arithmetic Mean ± SD (% CV) |

Total molybdenum

| | | | | | |
|---|---|---|---|---|---|
| Cₘₐₓ_{_{—}}ₙ (ng/mL)/(mg) | 74.0 ± 35.1 | 94.2 ± 47.4 | 89.9 ± 24.9 | 73.2 ± 21.8 | 59.5 ± 28.6 |
| | (47.5) | (50.3) | (27.7) | (29.8) | (48.0) |
| AUCₜ_{_{—}}ₙ (h*ng/mL)/(mg) | 3023.2 ± 1326.9 | 3655.2 ± 1444 | 3354 ± 997.8 | 2800.2 ± 906.0 | 2521.5 ± 707.5 |
| | (43.9) | (39.5) | (29.7) | (32.4) | (28.1) |
| AUC_{∞}_{_{—}}ₙ (h*ng/mL)/(mg) | NA | 4436.9 ± 933.5 | 4083.5 ± 606.8 | 3168.8 ± 870.1 | 2681.4 ± 826.1 |
| | | (21.0) | (14.9) | (27.5) | (30.8) |

PUF molybdenum

| | | | | | |
|---|---|---|---|---|---|
| Cₘₐₓ_{_{—}}ₙ (ng/mL)/(mg) | 13.0 ± 17.2 | 6.8 ± 11.3 | 6.8 ± 9.5 | 3.3 ± 1.3 | 3.7 ± 1.6 |
| | (131.9) | (167.0) | (138.6) | (37.9) | (44.7) |
| AUCₜ_{_{—}}ₙ (h*ng/mL)/(mg) | 691.9 ± 817.7 | 505.4 ± 1054.2) | 213.9 ± 201.4 | 120.8 ± 58.2 | 82.7 ± 17.2 |
| | (118.1) | (208.6) | (94.1) | (48.2) | (20.8) |

^{a}PK parameters were calculated based on corrected concentrations and all parameter values are rounded to one digit after decimal point from the source data.

The results of the analyses for a potential formulation difference between Treatments A and B indicate that there were no clinically meaningful differences in BC-TTM PK parameters between the 2 treatments or formulations. Plasma total molybdenum (Cₘₐₓ, AUCₜ, and AUC_{∞}) and PUF molybdenum (Cₘₐₓ) geometric mean ratios (90% CI) were contained entirely within the default no-effect 90% CI boundary of 80% to 125%, except for PUF molybdenum AUCₜ where geometric mean ratio (90% CI) was 101.2% (70.6% to 145.1%), with the lower and upper boundary marginally extending outside of the no-effect boundary of 80% to 125% (Table 19).

TABLE 19
Relative Bioavailability of Plasma Total and PUF Molybdenum (PKDS-CO Set)

| | Category | | | | | |
|---|---|---|---|---|---|---|
| | Geometric LSM | | | | | |
| | Treatment A | Treatment B | ANOVA-Derived | | | |
| | BC-TTM | BC-TTM | Geometric | | | |
| | 12 × 1.25 | 1 × 15 | Means Ratio^{b} | | | |
| | mg EC | mg EC | Ratio (%) | | Intra- | Inter- |
| PK | mini-tablets | tablet | (Test/ | 90% | Participant | Participant |
| Parameter^{a} | (Test) | (Reference) | Reference) | CI | CV (%) | CV (%) |

Plasma Total Molybdenum

| | | | | | | |
|---|---|---|---|---|---|---|
| Cₘₐₓ | 227.4 | 239.2 | 95.0 | 82.1- | 42.0 | 60.2 |
| (ng/mL) | | | | 110.0 | | |
| AUCₜ | 8835.1 | 9307.8 | 94.9 | 81.6- | 43.7 | 61.1 |
| (h*ng/mL) | | | | 110.5 | | |
| AUC_{∞} | 10267.1 | 10364.0 | 99.1 | 89.3- | 21.4 | 61.2 |
| (h*ng/mL) | | | | 109.9 | | |

PUF Molybdenum

| | | | | | | |
|---|---|---|---|---|---|---|
| Cₘₐₓ | 10.2 | 10.2 | 99.4 | 87.2- | 36.5 | 63.6 |
| (ng/mL) | | | | 113.4 | | |
| AUCₜ | 327.3 | 323.4 | 101.2 | 70.6- | 114.8 | 56.5 |
| (h*ng/mL) | | | | 145.1 | | |

^{a}PK parameters were calculated using corrected concentrations.
^{b}Bioavailability was derived using an ANOVA statistical model with dosing period, treatment, and treatment sequence as the fixed effects and the participant as a random effect, using the natural logarithms of the data. Bioavailability was then defined as the ratio of the geometric means of PK parameter (Cₘₐₓ, AUCₜ, and AUC_{∞}) for the test (12 × 1.25 mg BC-TTM EC mini-tablets) over the reference (1 × 15 mg BC-TTM EC tablet) treatment.

Total molybdenum: For the 2.5 mg to 15 mg dose range and the 2.5 mg to 30 mg dose range, dose-proportionality criteria for Cₘₐₓ and AUCₜ were met as 90% CI slope values fell inside the critical intervals defined as ([1+In(0.5)/In(ρ), 1-In(2)In(ρ)]). However, for the 2.5 ma to 5 mg dose range and the 2.5 mg to 10 mg dose range, the dose-proportionality criteria for Cₘₐₓ and AUCₜ were not met. For AUC_{∞}, the dose-proportionality criterion was met only for the 2.5 mg to 10 mg dose range, but was not for the 2.5 mg to 15 mg dose range and the 2.5 mg to 30 mg dose range. Overall, the power model based dose-proportionality analysis results demonstrate that increases in total molybdenum exposure are generally dose proportional across the investigated dose range of 2.5 mg to 30 mg.

PUF molybdenum: The dose-proportionality criteria for Cₘₐₓ and AUCₜ values were not met for any dose range. The power model-based dose-proportionality analysis results demonstrate that increases in PUF molybdenum exposure were less than dose proportional across the investigated dose range of 2.5 mg to 30 mg due, most likely, to the much higher variability in the PUF molybdenum concentrations versus plasma total molybdenum.

TABLE 20
Power Model Assessment of Dose-Proportionality
of Plasma Total and PUF Molybdenum (PKDS-E
Set and Treatment A from PKDS-CO Set)

| | BC-TTM | Power Model | 90% CI | |
|---|---|---|---|---|
| | Dose | Estimate | Lower | Upper |
| PK Parameter^{a} | Range^{b} | (slope) | limit | limit |

Total molybdenum

| | | | | |
|---|---|---|---|---|
| Cₘₐₓ | 2.5 mg to 5 mg | 1.255 | −0.485 | 2.996 |
| (ng/mL) | 2.5 mg to 10 mg | 1.257 | 0.504 | 2.011 |
| | 2.5 mg to 15 mg* | 1.041 | 0.721 | 1.361 |
| | 2.5 mg to 30 mg* | 0.977 | 0.733 | 1.222 |
| AUCₜ | 2.5 mg to 5 mg | 1.242 | 0.071 | 2.414 |
| (h*ng/mL) | 2.5 mg to 10 mg | 1.118 | 0.598 | 1.637 |
| | 2.5 mg to 15 mg* | 0.945 | 0.696 | 1.194 |
| | 2.5 mg to 30 mg* | 0.931 | 0.739 | 1.123 |
| AUC_{∞} | 2.5 mg to 5 mg | NA | NA | NA |
| (h*ng/mL) | 2.5 mg to 10 mg* | 0.825 | 0.345 | 1.305 |
| | 2.5 mg to 15 mg | 0.613 | 0.286 | 0.940 |
| | 2.5 mg to 30 mg | 0.575 | 0.401 | 0.749 |

PUF molybdenum

| | | | | |
|---|---|---|---|---|
| Cₘₐₓ | 2.5 mg to 5 mg | −0.137 | −1.793 | 1.520 |
| (ng/mL) | 2.5 mg to 10 mg | 0.477 | −0.360 | 1.314 |
| | 2.5 mg to 15 mg | 0.505 | 0.204 | 0.806 |
| | 2.5 mg to 30 mg | 0.599 | 0.339 | 0.859 |
| AUCₜ | 2.5 mg to 5 mg | −0.701 | −3.225 | 1.822 |
| (h*ng/mL) | 2.5 mg to 10 mg | 0.079 | −1.129 | 1.288 |
| | 2.5 mg to 15 mg | 0.220 | −0.288 | 0.728 |
| | 2.5 mg to 30 mg | 0.324 | −0.071 | 0.718 |

*Dose proportionality criteria was met as the 90% CI values were contained entirely within the critical interval defined as ([1 + In(0.5)/In(ρ), 1 + In(2)/In(ρ)]), dose-proportionality was supported across the investigated dose range.
^{a}PK parameters were calculated based on corrected concentrations.
^{b}Equivalent molybdenum dose was used in the power model dose-proportionality analysis.

There were no apparent differences in BC-TTM PD parameters (plasma total and PUF copper concentrations) between 12×1.25 mg EC mini-tablets and the 15 mg reference EC tablet. Maximum plasma total copper concentration occurred 8 hours post-dose and then gradually decreased and eventually returned to pre-dose Baseline concentrations by 96 to 120 hours post-dose. The pre-dose Baseline mean plasma total copper concentration of Treatments A and B were 988 and 986 ng/mL, respectively, and transiently increased to a mean maximum of 1230 and 1210 ng/mL, respectively, at 8 hours post-dose.

After the 8-hour post-dose time point, plasma total copper concentrations gradually decreased, and the mean concentration declined to <11% above the pre-dose Baseline at 48 hours post-dose. By 96 to 120 hours post-dose, total copper concentrations had returned to pre-dose Baseline concentrations. PUF copper concentrations were much lower than total copper concentrations (mean value of less than 10 ng/mL) at all sampling time points limiting the opportunity for quantitative assessments.

Summary statistics for absolute and percentage change from Baseline plasma total copper concentrations following Treatments C, D, E, and F were calculated. Treatment A from the Two-way Crossover Period was included for comparison. For Treatment C (2.5 mg BC-TTM, lowest BC-TTM dose), plasma total copper concentration versus time profiles remained stable overall. The plasma total copper concentration versus time profiles following Treatments D, E, and F showed a similar trend as the profiles of Treatments A and B. Plasma total copper concentrations reached a maximum at 6 to 12 hours post-dose and centered around 8 hours, with a maximum mean percentage change (increase) from Baseline (0.5 hours pre-dose) of approximately 2%, 10%, 18%, 26%, and 31% for Treatments C, D, E, A, and F, respectively. The increases of maximum mean percentage changes are dose dependent, but less than dose proportional.

After the 12-hour post-dose time point, plasma total copper concentrations gradually decreased with the median percent change from Baseline reaching within approximately <15% of the pre-dose Baseline at 48 hours post-dose. At 120 to 144 hours post-dose, total copper concentrations had returned to pre-dose Baseline levels, PUF copper concentrations were much lower than total copper concentrations (mean value of less than 10 ng/mL) at all sampling time points, limiting the opportunity for quantitative assessments

BC-TTM had an acceptable safety profile and was generally well-tolerated in healthy adult participants when administered as a single oral dose from 2.5 mg to 30 mg as EC mini-tablets and as a 15 mg EC tablet with no notable differences in the incidence of TEAEs. No deaths or TESAEs were reported. All TEAEs were Grade 1 or 2 in severity, except for 2 events of increased blood creatine phosphokinase blood concentrations of Grade 4 severity reported by 2 (4.3%) participants following Treatment B during the Two-way Crossover Period. The incidence of TEAEs was similar between Treatment A (BC-TTM 12×1.25 mg EC mini-tablets) and Treatment B (BC-TTM single 15 mg EC reference tablet), and no dose-relationship was observed for the Treatments C to F (2.5 mg to 30 mg BC-TTM administered as 1,25 mg EC mini-tablets).

### Example 6. Food Vehicle Study

The food study was performed to observe and test the integrity and stability of the BC-TTM 1.25-mg mini-tablets once introduced to a food vehicle. The BC-TTM 1.25-mg mini-tablets were prepared in accordance with Example 3 and the drug product batch formula of Table 8. The mini-tablets were tested at a 5-mg (4×1.25-mg) dose and a 1.25-mg dose in either yogurt or applesauce. The samples were allowed to soak in the food vehicles for allotted time-points at both room temperature and 5° C. food storage conditions. The samples were then removed from the food vehicles for visual observations and tested.

Specifically, the study was conducted as follows:
1. Samples were tested at n=3.
2. Both room temperature and 5° C. storage conditions of the food vehicles were tested to determine if the storage of the food vehicle had an influence on the integrity of the sample.
3. Samples were tested at a 5-mg dose (4×1.25-mg) and a 1.25-mg dose.
4. Applesauce Soaking Time-Points: 5, 7.5, 10, 12.5, and 15 minutes.
5. Yogurt Soaking Time-Points: 5, 10, 15, 30, 45, 60, 90, and 120 minutes,
6. Delivery technique: mini-tablets were placed on top of the food vehicle and stirred in from top to bottom a total of three times to best represent the handling likely during administration.
7. Food vehicles were not added to dissolution vessels following sample soaking.

### Yogurt

Yoplait Original French Vanilla Low Fat Yogurt (6 oz) (pH 4.24) was the brand used for the yogurt food vehicle.

A 5-mg dose (4×1.25-mg) or a 1.25-mg dose was placed on top of the yogurt and a spoon was used to stir in the mini-tablet(s) from bottom to top a total of three times, ensuring the samples were fully covered, The spoon was then removed and the foil-lid was placed over to cover. The mini-tablets were allowed to soak in the yogurt for the following time-points: 5, 10, 15, 30, 45, 60, 90, and 120 minutes. The samples for each time-point were tested at n=3, at both 5° C. and room temperature food vehicle storage conditions. The room temperature samples were left on the lab countertops for the duration of the food soaking, whereas the 5° C. samples were immediately placed into 5° C. storage after introduction to the yogurt. After the allotted time-points, the samples were removed from the yogurt and observed.

For the 5 mg dose, the mini-tablets were placed into dissolution apparatus 1 baskets and transferred to an acid stage bath (500 mL, 0.1 N HCl, 37° C.±0,5° C.) for two hours set to a rotation speed of 100 rpm. The samples were then removed from the acid bath for observation and transferred to a buffer stage bath (500 mL, modified Simulated Intestinal Fluid pH 7.5±0.05, 37° C.±0.5° C.) set to a rotation speed of 75 rpm. Samples were taken at 10, 12.5, 15, 20, and 30 minutes. Following the 20 minutes sampling time-point, the rotation speed was increased to 250 rpm. The samples were then analyzed using HPLC.

For the 1.25 mg dose, the mini-tablet was placed into a dissolution apparatus 2 mini-vessel acid stage bath (75 mL, 0.1 N HCl 37° C.±0.5° C.) for two hours set to a rotation speed of 100 rpm. Following the two-hour acid stage, the mini-tablet was observed and a buffer solution was added to the vessel (25 mL, 0.25M Tribasic Sodium Phosphate, pre-heated to 37° C.±0.5° C.). The paddle speed rotation was decreased to 75 rpm, and samples were taken at 10, 12.5, 15, 20, and 30 minutes. Following the 20 minutes sampling time-point, the rotation speed was increased to 250 rpm. The samples were then analyzed using HPLC.

For the 5-mg dose (4×1.25-mg), the samples tested in yogurt showed no visible signs of swelling or discoloration throughout testing. The integrity of the mini-tablets was not compromised by the introduction to yogurt.

For the 1.25 mg dose, the samples tested in yogurt showed no visible signs of swelling or discoloration throughout testing. The integrity of the mini-tablets coating was not compromised by the introduction to yogurt.

### Applesauce

Mott's Applesauce (4oz) (pH 3.68) was the brand used for the applesauce food vehicle.

A 5-mg dose (4×1.25-mg) or 1.25-mg dose was placed on top of the applesauce and a spoon was used to stir in the mini-tablets from bottom to top a total of three times, ensuring the samples were fully covered. The spoon was then removed and the foil-lid was placed over to cover. The mini-tablets were avowed to soak in the applesauce for the following time-points: 5, 7,5, 10. 12.5, and 15 minutes. The samples for each time-point were tested at n=3, at both 5° C. and room temperature food vehicle storage conditions, The room temperature samples were left on the lab countertops for the duration of the food soaking, whereas the 5° C. samples were immediately placed into 5° C. storage after introduction to the applesauce. After the allotted time-points, the samples were removed from the food vehicle and observed.

For the 5 mg dose, the mini-tablets were placed in dissolution apparatus 1 baskets and transferred to an acid stage bath (500 mL, 0.1 N HCl, 37° C. ±0.5° C.) for two hours set to a rotation speed of 100 rpm. The samples were then removed from the acid bath for observation and transferred to a buffer stage bath (500 mL, modified Simulated Intestinal Fluid pH 7.5±0.05, 37° C.±0.5° C.) set to a rotation speed of 75 rpm. Samples were taken at 10, 12.5, 15, 20, and 30 minutes. Following the 20 minutes sampling time-point, the rotation speed was increased to 250 rpm. The samples were then analyzed using HPLC,

For the 1.25 mg dose, the mini-tablet was placed into a dissolution apparatus 2 mini-vessel acid stage bath (75 mL, 0.1 N HCl 37° C.±0.5° C.) for two hours set to a rotation speed of 100 rpm. Following the two-hour acid stage, the mini-tablet was observed and a buffer solution was added to the vessel (25 mL, 0.25M Tribasic Sodium Phosphate pre-heated to 37° C.±0.5° C.). The paddle speed rotation was decreased to 75 rpm, and samples were taken at 10, 12.5, 15, 20, and 30 minutes. Following the 20-minute sampling time-point, the rotation speed was increased to 250 rpm. The samples were then analyzed using HPLC,

For the 5 mg does, following the soaking in the applesauce, the mini-tablets were observed and there were no visible signs of discoloration or degradation. All samples were then moved to the two-hour acid stage bath. Following the two-hour acid stage, the mini-tablets were observed. All 5° C. time-point samples remained intact, with no signs of swelling or discoloration. The room temperature 5 and 7.5 minute time-point samples also remained intact, with no visible signs of discoloration or degradation. All the room temperature time-point samples following 7.5 minutes (10, 12.5, and 15 minutes) had degraded in the acid stage. Only the 5° C. samples and the 5 and 7.5 minute time-point room temperature samples were able to continue to the buffer stage,

For the 1.25 mg does, following the soaking in the applesauce, the mini-tablet was observed and there was no visible sign of discoloration or degradation. All samples were then moved to the two-hour acid stage bath. Following the two-hour acid stage, the mini-tablets were observed. All 5° C. time-point samples remained intact, with no signs of swelling or discoloration. The room temperature 5 and 7.5 minute time-point samples also remained intact, with no visible signs of swelling or degradation. All the room temperature time-point samples following 7.5 minutes (10, 12.5, and 15 minutes) showed signs of slight swelling throughout the acid stage, but no visible signs of discoloration or degradation.

The results summarized in this example confirm that BC-TTM 1.25-mg enteric coated mini-tablets have stability after introduction to a food vehicle. In applesauce, the mini-tablets are stable for up to 7.5 minutes at room temperature, and up to 15 minutes at 5° C. (refrigerated) for 5-mg doses (4×1.25-mg) and up to 15 minutes at both room temperature and 5° C. (refrigerated) storage conditions for 1.25-ma doses. In yogurt, the mini-tablets are stable for up to 120 minutes at both room temperature and 5° C. (refrigerated) storage conditions for 5-mg (4×1.25-mg) and 1.25-mg doses.

It is understood that the examples and embodiments described herein are for illustrative purposes only and that various modifications or changes in light thereof are suggested to persons skilled in the art and are to be incorporated within the spirit and purview of this application and scope of the appended claims. All publications, patents, and patent applications cited herein are hereby incorporated herein by reference for all purposes.

What is claimed is:

1. A mini-tablet formulation comprising bis-choline tetrathiomolybdate in an amount in the range of about 1.00 mg to about 1.50 mg (e.g., in the range of about 1.10 mg to about 1,40 mg, or about 1.15 mg to about 1.35 mg, or about 1.20 mg to about 1.30 mg, or about 1.22 mg to about 1.28 mg, or about 1.23 mg to about 1.27 mg, or about 1.24 mg to about 1.26 mg).

2. The mini-tablet formulation of claim 1, wherein the amount of bis-choline tetrathiomolybdate is about 1.25 mg.

3. The mini-tablet formulation of claim 1 or claim 2, further comprising about 20% to about 30% (e.g., in the range of about 22% to about 28%, or about 23% to about 27%, or about 24% to about 26%, or about 20% to about 25%, or about 25% to about 30%) by weight, based on the weight of mini-tablet core, of a buffer.

4. The mini-tablet formulation of claim 1 or claim 2, further comprising about 25 wt %, based on the weight of mini-tablet core, of a buffer.

5. The mini-tablet formulation of claim 3 or 4, wherein the buffer is sodium bicarbonate.

6. The mini-tablet formulation of any one of claims 1-5, further comprising about 60% to about 70% (e.g., in the range of about 62% to about 70%, or about 63% to about 69%, or about 64% to about 68%, or about 65% to about 67%) by weight, based on the weight of mini-tablet core, of a filler component.

7. The mini-tablet formulation of claim 6, further comprising about 66 wt %, based on the weight of mini-tablet core, of a filler component.

8. The mini-tablet formulation of claim 6 or 7, wherein the filler component is microcrystalline cellulose.

9. The mini-tablet formulation of any one of claims 1-8, further comprising about 0.5% to about 1% (e.g., in the range of about 0.6% to about 0,9%, or about 0.65% to about 0.85%, or about 0.7% to about 0.8%, or about 0.72% to about 0.78%, or about 0.73% to about 0.77%) by weight, based on the weight of mini-tablet core, of a lubricant component.

10. The mini-tablet formulation of claim 9, further comprising about 0.75% of the lubricant component.

11. The mini-tablet formulation of claim 9 or 10, wherein the lubricant component is sodium stearyl furnarate.

12. The mini -tablet formulation of any one of claims 1-11 further comprising a coating on the outer surface of the formulation (e.g., an outer surface of the mini-table's core that comprises bis-choline tetrathiomolybdate and optionally the buffer, the filler component, and/or the lubricant component).

13. The mini -tablet formulation of claim 12, wherein the coating comprises a seal coating, a sub-coating, an enteric coating, or a combination thereof.

14. A mini-tablet formulation comprising:
bis-choline tetrathiomolybdate in an amount of about 1.25 mg;
about 25% (by weight based on the weight of mini-tablet core) of a buffer;
about 66% (by weight based on the weight of mini-tablet core) of a filler component;
about 0.75% (by weight based on the weight of mini-tablet core) of the lubricant component.

15. The mini-tablet formulation of claim 14, further comprising a coating on an outer surface of the mini-tablet's core that comprises bis-choline tetrathiomolybdate, the buffer, the filler component, and the lubricant component.

16. The mini-tablet formulation of claim 15, wherein the coating comprises a seal coating, a sub-coating, an enteric coating, or a combination thereof.

17. The mini-tablet formulation of any one of claims 14-16, wherein buffer is sodium bicarbonate.

18. The mini-tablet formulation of any of claims 14-17, wherein the filler component is microcrystalline cellulose.

19. The mini-tablet formulation of any of claims 14-18, wherein the lubricant component is sodium stearyl fumarate.

20. The mini-tablet formulation of any one of claims 1-19, wherein the mini-tablet formulation comprises no more than about 3% of total impurities at 4 weeks of storage at about 25° C. at about 60% relative humidity.

21. The mini-tablet formulation of any one of claims 1-19, wherein the mini-tablet formulation comprises less than about 2%, of total molybdenum impurities, wherein the molybdenum impurities are selected from one or more of TM0, TM1, TM2, and TM3, at 4 weeks of storage at about 25° C. at about 60% relative humidity.

22. The mini-tablet formulation of any one of claims 1-19, wherein the mini-tablet formulation comprises no more than about 0.7% of polymeric molybdenum impurities.

23. The mini-tablet formulation of any one of claims 1-19, wherein the mini-tablet formulation comprises less than about 1.3% of TM3 impurity at 4 weeks of storage at about 25° C. at about 60% relative humidity.

24. The mini-tablet formulation of any one of claims 1-19, wherein the mini-tablet formulation comprises less than about 0.3% of Dimer S6 impurity at 4 weeks of storage at about 25° C. at about 60% relative humidity.

25. A unit dose container comprising one or more of the mini-tablets of any claims 1-24.

26. The unit dose container of claim 25 comprising from 2 to 24 mini-tablets.

27. The unit dose container of claim 26, comprising 2, 4, 8, 12, or 24 mini-tablets.

28. The unit dose container of any one of claims 25-27 comprising a capsule that can be opened by the patient, a sachet, or a stick pack.

29. The container of any one of claims 25-27 comprising a unit dose dispenser configured to dispense a unit dose of mini-tablets.

30. The unit dose container of claim 29, wherein the unit dose dispenser is a mini-tablet dispenser.

31. The unit dose container of claim 30, wherein the dispenser is configured to dispense about 2 to 24 mini-tablets.

32. The unit dose container of claim 31, wherein the dispenser is configured to dispense a unit dose of 2, 4, 8, 12, or 24 mini-tablets.

33. A method for treating a copper metabolism-associated disease or disorder in a subject, the method comprising administering to the subject one or more mini-tablets of any claims 1-24 or a unit dose as described in any of claims 25-32.

34. The method of claim 33, wherein the copper metabolism-associated disease or disorder is Wilson Disease.

35. The method of claim 33 or claim 34, wherein the one or more mini-tablets or the unit dose is administered daily, optionally once daily.

36. The method of claim 33 or claim 34, wherein the one or more mini-tablets or the unit dose is administered every other day.

37. The method of any claims 33-36, wherein the one or more mini-tablets or the unit dose is administered in fasted state.

38. The method of any claims 33-37, wherein the amount of bis-choline tetrathiomolybdate administered is 15 mg.

39. Use of one or more mini-tablets of any claims 1-24 or a unit dose as described in any of claims 25-32 for the manufacture of a medicament.

40. Use of one or more mini-tablets of any claims 1-24 or a unit dose as described in any of claims 25-32 for the manufacture of a medicament for treating a copper metabolism-associated disease or disorder in a subject.

41. The use of claim 40, wherein the copper metabolism-associated disease or disorder is Wilson Disease.

42. The use of any of claims 39-41, wherein the one or more mini-tablets or the unit dose is administered daily, optionally once daily.

43. The use of any of claims 39-41, wherein the one or more mini-tablets or the unit dose is administered every other day.

44. The use of any of claims 39-43, wherein the one or more mini-tablets or the unit dose is administered in fasted state.

45. , The use of any of claims 39-44, wherein the amount of bis-choline tetrathiamolybdate administered is 15 mg.
